SEQUENCE LISTING (11 sequences)

SEQ ID NO: 1 - DNA, 1743 bp, Homo sapiens
atgagcagctgcaggtacaacgggggctgcatgcggccgctcagcaacttgagcgcgtcccgccggaacctccacgagatggactcagaggcgcagcccctgcagccccccgcgtctgtcggaggaggtggcggcgcgtcctccccgtctgcagccgctgccgccgccgccgccgctgtttcgtcctcagcccccgagatcgtggtgtctaagcccgagcacaacaactccaacaacctggcgctctatggaaccggcggcggaggcagcactggaggaggcggcggcggtggcgggagcgggcacggcagcagcagtggcaccaagtccagcaaaaagaaaaaccagaacatcggctacaagctgggccaccggcgcgccctgttcgaaaagcgcaagcggctcagcgactacgcgctcatcttcggcatgttcggcatcgtggtcatggtcatcgagaccgagctgtcgtggggcgcctacgacaaggcgtcgctgtattccttagctctgaaatgccttatcagtctctccacgatcatcctgctcggtctgatcatcgtgtaccacgccagggaaatacagttgttcatggtggacaatggagcagatgactggagaatagccatgacttatgagcgtattttcttcatctgcttggaaatactggtgtgtgctattcatcccatacctgggaattatacattcacatggacggcccggcttgccttctcctatgccccatccacaaccaccgctgatgtggatattattttatctataccaatgttcttaagactctatctgattgccagagtcatgcttttacatagcaaacttttcactgatgcctcctctagaagcattggagcacttaataagataaacttcaatacacgttttgttatgaagactttaatgactatatgcccaggaactgtactcttggtttttagtatctcattatggataattgccgcatggactgtccgagcttgtgaaaggtaccatgatcaacaggatgttactagcaacttccttggagcgatgtggttgatatcaataactttcctctccattggttatggtgacatggtacctaacacatactgtggaaaaggagtctgcttacttactggaattatgggtgctggttgcacagccctggtggtagctgtagtggcaaggaagctagaacttaccaaagcagaaaaacacgtgcacaatttcatgatggatactcagctgactaaaagagtaaaaaatgcagctgccaatgtactcagggaaacatggctaatttacaaaaatacaaagctagtgaaaaagatagatcatgcaaaagtaagaaaacatcaacgaaaattcctgcaagctattcatcaattaagaagtgtaaaaatggagcagaggaaactgaatgaccaagcaaacactttggtggacttggcaaagacccagaacatcatgtatgatatgatttctgacttaaacgaaaggagtgaagacttcgagaagaggattgttaccctggaaacaaaactagagactttgattggtagcatccacgccctccctgggctcataagccagaccatcaggcagcagcagagagatttcattgaggctcagatggagagctacgacaagcacgtcacttacaatgctgagcggtcccggtcctcgtccaggaggcggcggtcctcttccacagcaccaccaacttcatcagagagtagctag

SEQ ID NO: 2 - DNA, 1740 bp, Homo sapiens
atgagcagctgcaggtacaacgggggcgtcatgcggccgctcagcaacttgagcgcgtcccgccggaacctccacgagatggactcagaggcgcagcccctgcagccccccgcgtctgtcggaggaggtggcggcgcgtcctccccgtctgcagccgctgccgccgccgccgctgtttcgtcctcagcccccgagatcgtggtgtctaagcccgagcacaacaactccaacaacctggcgctctatggaaccggcggcggaggcagcactggaggaggcggcggcggtggagggagcgggcacggcagcagcagtggcaccaagtccagcaaaaagaaaaaccagaacatcggctacaagctgggccaccggcgcgccctgttcgaaaagcgcaagcggctcagcgactacgcgctcatcttcggcatgttcggcatcgtggtcatggtcatcgagaccgagctgtcgtggggcgcctacgacaaggcgtcgctgtattccttagctctgaaatgccttatcagtctctccacgatcatcctgctcggtctgatcatcgtgtaccacgccagggaaatacagttgttcatggtggacaatggagcagatgactggagaatagccatgacttatgagcgtattttcttcatctgcttggaaatactggtgtgtgctattcatcccatacctgggaattatacattcacatggacggcccggcttgccttctcctatgccccatccacaaccaccgctgatgtggatattattttatctataccaatgttcttaagactctatctgattgccagagtcatgcttttacatagcaaacttttcactgatgcctcctctagaagcattggagcacttaataagataaacttcaatacacgttttgttatgaagactttaatgactatatgcccaggaactgtactcttggtttttagtatctcattatggataattgccgcatggactgtccgagcttgtgaaaggtaccatgatcaacaggatgttactagcaacttccttggagcgatgtggttgatatcaataacttttctctccattggttatggtgacatggtacctaacacatactgtggaaaaggagtctgcttacttactggaattatgggtgctggttgcacagccctggtggtagctgtagtggcaaggaagctagaacttaccaaagcagaaaaacacgtgcacaatttcatgatggatactcagctgactaaaagagtaaaaaatgcagctgccaatgtactcagggaaacatggctaatttacaaaaatacaaagctagtgaaaaagatagatcatgcaaaagtaagaaaacatcaacgaaaattcctgcaagctattcatcaattaagaagtgtaaaaatggagcagaggaaactgaatgaccaagcaaacactttggtggacttggcaaagacccagaacatcatgtatgatatgatttctgacttaaacgaaaggagtgaagacttcgagaagaggattgttaccctggaaacaaaactagagactttgattggtagcatccacgccctccctgggctcataagccagaccatcaggcagcagcagagagatttcattgaggctcagatggagagctacgacaagcacgtcacttacaatgctgagcggtcccggtcctcgtccaggaggcggcggtcctcttccacagcaccaccaacttcatcagagagtagctag

SEQ ID NO: 3 - protein, 580 aa, Homo sapiens
MSSCRYNGGVMRPLSNLSASRRNLHEMDSEAQPLQPPASVGGGGGASSPSAAAAAAAAAVSSSAPEIVVSKPEHNNSNNLALYGTGGGGSTGGGGGGGGSGHGSSSGTKSSKKKNQNIGYKLGHRRALFEKRKRLSDYALIFGMFGIVVMVIETELSWGAYDKASLYSLALKCLISLSTIILLGLIIVYHAREIQLFMVDNGADDWRIAMTYERIFFICLEILVCAIHPIPGNYTFTWTARLAFSYAPSTTTADVDIILSIPMFLRLYLIARVMLLHSKLFTDASSRSIGALNKINFNTRFVMKTLMTICPGTVLLVFSISLWIIAAWTVRACERYHDQQDVTSNFLGAMWLISITFLSIGYGDMVPNTYCGKGVCLLTGIMGAGCTALVVAVVARKLELTKAEKHVHNFMMDTQLTKRVKNAAANVLRETWLIYKNTKLVKKIDHAKVRKHQRKFLQAIHQLRSVKMEQRKLNDQANTLVDLAKTQNIMYDMISDLNERSEDFEKRIVTLETKLETLIGSIHALPGLISQTIRQQQRDFIEAQMESYDKHVTYNAERSRSSSRRRRSSSTAPPTSSESS

SEQ ID NO: 4 - protein, 579 aa, Homo sapiens
MSSCRYNGGVMRPLSNLSASRRNLHEMDSEAQPLQPPASVGGGGGASSPSAAAAAAAAVSSSAPEIVVSKPEHNNSNNLALYGTGGGGSTGGGGGGGGSGHGSSSGTKSSKKKNQNIGYKLGHRRALFEKRKRLSDYALIFGMFGIVVMVIETELSWGAYDKASLYSLALKCLISLSTIILLGLIIVYHAREIQLFMVDNGADDWRIAMTYERIFFICLEILVCAIHPIPGNYTFTWTARLAFSYAPSTTTADVDIILSIPMFLRLYLIARVMLLHSKLFTDASSRSIGALNKINFNTRFVMKTLMTICPGTVLLVFSISLWIIAAWTVRACERYHDQQDVTSNFLGAMWLISITFLSIGYGDMVPNTYCGKGVCLLTGIMGAGCTALVVAVVARKLELTKAEKHVHNFMMDTQLTKRVKNAAANVLRETWLIYKNTKLVKKIDHAKVRKHQRKFLQAIHQLRSVKMEQRKLNDQANTLVDLAKTQNIMYDMISDLNERSEDFEKRIVTLETKLETLIGSIHALPGLISQTIRQQQRDFIEAQMESYDKHVTYNAERSRSSSRRRRSSSTAPPTSSESS

SEQ ID NO: 5 - DNA, 39 bp, Artificial Sequence
  Description of Artificial Sequence Synthetic primer
acgatgaattcgccaccatgagcagctgcaggtacaacg

SEQ ID NO: 6 - DNA, 38 bp, Artificial Sequence
  Description of Artificial Sequence Synthetic primer
acgactactcgagctagctactctctgatgaagttggt

SEQ ID NO: 7 - DNA, 24 bp, Artificial Sequence
  Description of Artificial Sequence Synthetic primer
tggactgtccgagcttgtgaaagg

SEQ ID NO: 8 - DNA, 23 bp, Artificial Sequence
  Description of Artificial Sequence Synthetic primer
ccttggtggtagccgtagtggca

SEQ ID NO: 9 - DNA, 25 bp, Artificial Sequence
  Description of Artificial Sequence Synthetic primer
tgagcactggggagaaaggatttgg

SEQ ID NO: 10 - DNA, 25 bp, Artificial Sequence
  Description of Artificial Sequence Synthetic primer
tcggagatggtgatcttcttgctgg

SEQ ID NO: 11 - DNA, 106 bp, Artificial Sequence
  Description of Artificial Sequence Synthetic probe
agcccccagcgtcggttgtaggaggaggtggtggtgcgtcctccccgtctgctgccgccgccgcctcatcctcagccccagagatcgtggtgtctaagccggagca

(19) United States
(12) Patent Application Publication
(10) Pub. No.: US 2008/0268439 A1
(43) Pub. Date: Oct. 30, 2008

(54) ASSAY SYSTEMS AND METHODS FOR DETECTING MOLECULES THAT INTERACT WITH SK2 CHANNELS

(76) Inventors: Edward Kaftan, Pennington, NJ (US); Adrienne Elizabeth Dubin, San Diego, CA (US); Sandra R. Chaplan, San Diego, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/739,789

(22) Filed: Apr. 25, 2007

(51) Int. Cl. C12Q 1/68; G01N 33/53

(52) U.S. Cl. 435/6; 435/7.1

(57) ABSTRACT

The invention provides methods including steps of: providing cells capable of expressing SK2; contacting the cells with a test molecule; obtaining information indicative of cellular SK2 expression to obtain an SK2 Expression Value; comparing the SK2 Expression Value with a control SK2 Expression Value; and identifying a test molecule that causes the cells to display an SK2 Expression Value that is different from the control SK2 Expression Value. Also provided are methods including steps of: providing a sample comprising an SK2 channel; contacting the sample with a test molecule; obtaining information indicative of SK2 channel activity in the sample to obtain an SK2 Channel Activity Value; comparing the SK2 Channel Activity Value with a control Channel Activity Value; and identifying a test molecule that causes the SK2 Channel Activity Value to be different from the control Channel Activity Value. Methods of identifying a molecule useful for treating neuropathic pain are also described.

The present invention claims priority from U.S. Provisional Patent Application 60/515,143 entitled “Assay systems and methods for detecting molecules that interact with SK2 channels” filed Oct. 28, 2003, the contents of which is hereby incorporated by reference in its entirety. The invention relates to expression of small-conductance calcium-activated potassium (SK) channels in neurons, as well as the role of SK channels in neuropathic pain.

### FIELD OF THE INVENTION

### Background of the Invention

Peripheral neuropathy (also referred to herein as “neuropathic pain”) is a neurological disorder resulting from damage or other trauma to the peripheral nerves. Many medical conditions include peripheral neuropathy amongst their manifestations, but peripheral neuropathy may also be an isolated finding. Nerve damage may be simple or multifactorial, and can be caused directly or indirectly by infection and consequent immune responses (for example Lyme disease, shingles (Varicella zoster), HIV, or as in post-polio syndrome), cancers (due to direct invasion, infiltration, pressure, or humoral influences), disorders of vascular supply including ischemia due to peripheral vascular disease, thromboembolism, infarction, collagen-vascular or other autoimmune diseases including systemic lupus erythematosus, scleroderma, sarcoidosis, rheumatoid arthritis, and vasculitis such as polyarteritis nodosa; metabolic/endocrine disorders such as diabetes, uremia, hyperthyroidism or hypothyroidism, and porphyria; storage diseases or diseases characterized by abnormal intracellular or extracellular accumulations such as amyloidosis, Gaucher's or multiple myeloma; trauma, including crush, penetrating injury, surgical division or irritation, traction or avulsion, contusion, fracture or dislocated bones, compression, entrapment, or pressure (e.g. carpal tunnel syndrome), intraneural hemorrhage; exposure to cold or radiation; toxins, including some medications such as cancer chemotherapeutic and antiviral drugs; some nutritional deficiencies; other inherited disorders, including von Recklinghausen's neurofibromatosis; and idiosyncratic causes including Guillain-Barre syndrome. In addition, peripheral neuropathies not uncommonly occur without an obvious medical cause.

Typically, the pain associated with peripheral neuropathy occurs or persists without an obvious noxious input. Although causes of peripheral neuropathy are diverse, common symptoms include weakness, numbness, paresthesias and dysesthesias (abnormal sensations such as burning, tickling, pricking or tingling), and pain in the arms, hands, legs, and/or feet. Some specific documented symptoms include hyperalgesia (extreme sensitivity to something painful), allodynia (something that does not ordinarily cause pain actually causes pain), and causalgia (persistent and extreme burning pain).

There is compelling evidence indicating that at least some hyperalgesia, allodynia and ongoing pain associated with peripheral nerve injury is due to changes in primary afferent neurons. Neuropathic pain reflects, at least in part, changes in the excitability and/or phenotype of primary afferent neurons. One particularly important change is the development of ongoing or ectopic activity in the neurons (also referred to herein as spontaneous discharge activity). Clinical observations indicate that ectopic activity of neurons contributes to ongoing neuropathic pain. Further, experimental evidence has been obtained that correlates the time course of behavioral changes in response to a spinal nerve ligation injury with that of the ectopic activity arising from the injured nerve. Data indicates that ectopic activity develops rapidly over the first one to three days post-injury, and then slowly declines over the subsequent weeks. This pattern of change in neural ectopic activity correlates with the pattern of changes in behavior. Spinal Nerve Ligation: What to Blame for the Pain and Why, Gold, M. S. (2000) Pain 84:117-120.

Firing of neurons is correlated to intracellular ion concentration. During a burst of action potentials, Ca²⁺ ions enter the neuron faster than a cell can clear them away. The intracellular concentration of Ca²⁺ increases during a high frequency burst of action potentials until the concentration of Ca²⁺ ions reaches the range in which Ca²⁺ binds to calcium-activated potassium channels, at which point the channels undergo conformation changes that result in channel activation. These calcium-activated potassium channels hyperpolarize the cell and tend to shut off activity and Ca²⁺ entry into the cell. Cessation of activity occurs during what is referred to as the afterhyperpolarization (AHP) of the membrane. As the Ca²⁺ load is cleared away, the intracellular concentration of Ca²⁺ ions decreases, and the calcium-activated potassium channels eventually shut. A new cycle of neuron bursting can then begin.

Thus, action potentials can be followed by a prolonged AHP of the membrane. Important functions of the AHP are to limit the number of action potentials and to slow down the firing frequency of neurons during sustained stimulations, a phenomenon known as “spike frequency adaptation.” The currents underlying the AHP are mediated by one type of calcium-activated potassium channel, known as small-conductance voltage-insensitive calcium-activated potassium channels (SK channels). SK channels are present in most neurons and play essential roles in regulating cellular functions by coupling intracellular Ca²⁺ levels and membrane potential to K⁺ efflux. The primary function of SK channels is to hyperpolarize nerve cells following one or several action potentials, in order to prevent long trains of epileptogenic activity from occurring.

To date, three main SK channels have been identified, SK1, SK2, and SK3. Among these channels, SK2 is expressed the most widely and abundantly in central neurons. SK channels are selectively blocked by apamin (an octadecapeptide from honey-bee venom) and have a small unitary conductance of 4-20 picosiemens (pS). An SK channel is a heteromer comprising multiple subunits of SK channel proteins and calmodulin complexes. An SK channel can be heteromeric, when it is composed of different SK channel protein subunits, or homomeric, when it is composed of the same SK channel protein subunits. For example, a homomeric SK2 channel is composed of SK2 protein subunits only, whereas a heteromeric SK2 channel is composed of SK2 channel protein combined with other SK channel proteins, such as SK1 and/or SK3.

### SUMMARY OF THE INVENTION

In one aspect, the invention provides novel assays and related methods to identify molecules that affect expression or function of an SK2 channel. In one such embodiment, the invention provides a method comprising steps of:
a. providing cells capable of expressing SK2;
b. contacting the cells with a test molecule;
c. obtaining information indicative of cellular SK2 expression to obtain an SK2 Expression Value;
d. comparing the SK2 Expression Value with a control SK2 Expression Value; and
e. identifying a test molecule that causes the cells to display an SK2 Expression Value that is different from the control SK2 Expression Value.
   In preferred embodiments, the step of identifying a test molecule comprises identifying a test molecule that causes the cells to display an SK2 Expression Value that is greater than the control SK2 Expression Value.

In another aspect, the invention provides a method comprising steps of:
a. providing a sample comprising a nucleic acid sequence having a gene under the control of an SK2 regulatory sequence;
b. contacting the sample with a test molecule;
c. obtaining information indicative of expression of the gene to obtain a gene Expression Value;
d. comparing the gene Expression Value with a control gene Expression Value; and
e. identifying a test molecule that causes the sample to display a gene Expression Value that is different from the control gene Expression Value.
   Preferably, the identifying step comprises identifying a test molecule that causes the sample to display a gene Expression Value that is greater than the control Expression Value.

In yet another aspect, the invention provides a method of identifying a molecule useful for treating neuropathic pain, the method comprising steps of:
a. providing cells capable of expressing SK2;
b. contacting the cells with a test molecule;
c. obtaining information indicative of SK2 cellular expression;
d. comparing the SK2 cellular expression in response to the test molecule with a control; and
e. identifying a test molecule useful for treating neuropathic pain as a molecule that causes cells to display an increase in the SK2 cellular expression relative to the control.

In another aspect, the invention provides a method comprising steps of:
a. providing a sample comprising an SK2 channel;
b. contacting the sample with a test molecule;
c. obtaining information indicative of SK2 channel activity in the sample to obtain an SK2 Channel Activity Value;
d. comparing the SK2 Channel Activity Value with a control Channel Activity Value; and
e. identifying a test molecule that causes the SK2 Channel Activity Value to be different from the control Channel Activity Value.
   Preferably, the identifying step comprises identifying a test molecule that causes the SK2 Channel Activity Value to be greater than the control Channel Activity Value.

In another aspect, the invention provides a method of identifying a molecule useful for treating neuropathic pain comprising steps of:
a. providing cells capable of expressing SK2;
b. contacting the cells with a membrane potential sensitive fluorescent dye;
c. contacting the cells with a test molecule;
d. obtaining information indicative of a change in membrane potential in response to the test molecule;
e. contacting the cells with a specific inhibitor of the SK2 channel; and
f. determining whether the change in membrane potential is blocked by the specific inhibitor.

In yet another aspect, the invention provides a method of identifying a molecule useful for treating neuropathic pain, the method comprising steps of:
a. providing a sample comprising an SK2 channel;
b. contacting the sample with a test molecule;
c. obtaining information indicative of SK2 channel activity in the sample to obtain an SK2 Channel Activity Value;
d. comparing the SK2 Channel Activity Value with a control Channel Activity Value; and
e. identifying a test molecule useful for treating neuropathic pain as a molecule that causes the SK2 Channel Activity Value to be greater than the control Channel Activity Value.

In another aspect, the invention provides a method of identifying a molecule useful for treating neuropathic pain, the method comprising steps of:
a. providing a sample comprising an SK2 channel;
b. contacting the sample with a test molecule;
c. obtaining information indicative of spontaneous discharge activity in the sample;
d. comparing the spontaneous discharge activity in the sample with a control; and
e. identifying a test molecule useful for treating neuropathic pain as a molecule that causes a decrease in the spontaneous discharge activity when the test molecule is present relative to the control.

In another aspect, a novel human isoform of SK2 has been discovered, which includes an additional alanine residue (Ala). In yet another aspect, the invention provides methods of hyperpolarizing a cell, as well as methods for creating a neuropathic pain model.

Still further aspects and embodiments of the invention will be described in more detail in the following figures and detailed description of the invention.

### BRIEF DESCRIPTION OF THE DRAWINGS

The accompanying drawings, which are incorporated in and constitute a part of this specification, illustrate several aspects of the invention and together with the description of the preferred embodiments, serve to explain the principles of the invention. A brief description of the drawings is as follows:

FIG. 1 is a graph illustrating SK2 mRNA expression levels (Y axis) at time periods (X axis) in DRG of spinal nerve ligated rats.

FIG. 2 is a nucleic acid sequence (SEQ ID NO: 1) that encodes a human SK2 isoform identified herein as hSK2A⁺.

FIG. 3 is the amino acid sequence (SEQ ID NO: 3) encoded by SEQ ID NO: 1.

FIG. 4 is a nucleic acid sequence (SEQ ID NO: 2) that encodes a human SK2 isoform identified herein as hSK2A⁻.

FIG. 5 is the amino acid sequence (SEQ ID NO: 4) encoded by SEQ ID NO: 2.

FIG. 6 illustrates function of hSK2A⁺isoform in mammalian tsA201 cells; current (pA) and voltage (mV) are represented on the Y-axis, and time (msec) is represented on the X-axis.

FIG. 7 illustrates the current required to clamp tsA201 cells expressing hSK2A⁺ at −25 mV; holding current at −25 mV is represented on the Y-axis (pA), and time (s, seconds) is represented on the X-axis.

FIG. 8 illustrates the activity of hSK2 measured in a mammalian cell using a membrane potential sensitive dye; fluorescence (au) is represented on the Y-axis, and time (seconds) is represented on the X-axis.

FIG. 9 illustrates pharmacological characterization of hSK2A⁺ expressed in mammalian tsA201 cells using a fluorescence assay that monitors membrane potential; concentration of test molecules is represented on the X-axis (log[compound]), and normalized activity is represented on the Y-axis.

FIG. 10 illustrates pharmacological characterization of hSK2A⁺ expressed in mammalian tsA201 cells using a fluorescence assay that monitors membrane potential, wherein the effect of an SK2 channel opener is observed; concentration of riluzole (log[riluzole]) is represented on the X-axis, and normalized activity is represented on the Y-axis.

### DETAILED DESCRIPTION OF THE INVENTION

The embodiments of the present invention described below are not intended to be exhaustive or to limit the invention to the precise forms disclosed in the following detailed description. Rather, the embodiments are chosen and described so that others skilled in the art can appreciate and understand the principles and practices of the present invention.

In a broad aspect, the present invention relates, at least in part, to the discovery of the role of small-conductance calcium-activated potassium (SK) channels in neuropathic pain. In the present invention, we show that expression of SK2 channels is decreased in neural cells of a neuropathic pain model as compared to a control. This indicates a possible role for the currents mediated by these channels in pain processing. This discovery can be utilized to identify, for example, compounds or mechanisms that may be involved in the regulation of neuropathic pain, as well as compounds that may be useful in treating neuropathic pain. The invention provides a new therapeutic target, SK2 channels, for developing novel methods and strategies for treatment of neuropathic pain. The use of SK2 channels as molecular targets for compounds to treat neuropathic pain, and the identification of modulators of SK2 channels for treatment of neuropathic pain are the subject of the present invention.

Because the invention relates to SK channels, some general concepts relating to such channels will be described in some detail. Generally, SK channels are membrane channels that are voltage-independent and open in response to an increase in the intracellular calcium concentration, [Ca²⁺], with an apparent K_{d} in the range of about 500 to about 1000 nM [Ca²⁺]ᵢ. The single channel conductance of SK channels is typically in the range of about 2 pS to about 20 pS. Typically, an SK channel is composed of multiple subunits of SK channel proteins and calmodulin complexes.

In turn, a small conductance, calcium-activated potassium channel protein, or “SK channel protein” (also referred to as an “SK protein”), refers to a polypeptide that is a subunit or monomer of an SK channel, and a member of the SK gene family (for example, SK1, SK2, SK3, and the like). An “SK gene” is a DNA molecule that encodes an SK channel protein, such as the genes encoding SK1, SK2, SK3 protein, or the like.

Various terms relating to the systems and methods of the invention are used throughout the specification.

In particular, the term “SK2 channel” refers to a membrane channel comprising an SK2 protein subunit. An SK2 channel can be heteromeric, when it is composed of SK2 channel protein combined with other SK channel proteins, such as SK1 or SK3, and calmodulin complexes. An SK2 channel can also be homomeric, when it is composed of SK2 channel protein and calmodulin complexes.

The term “SK2 protein” refers to a polypeptide that is a subunit or monomer of an SK2 channel, including, for example, polymorphic variants, alleles, mutants, or interspecies homologs that: (1) have a sequence that has greater than about 60% amino acid sequence identity, or about 65, 70, 75, 80, 85, 90, or 95% amino acid sequence identity, to a sequence of an SK2 protein, preferably a human SK2 protein as shown in SEQ ID NO: 4; or (2) bind to antibodies (such as polyclonal or monoclonal antibodies) raised against an immunogen comprising an SK2 protein, preferably a human SK2 protein as shown in SEQ ID NO: 4; or (3) encoded by a gene sequence that has greater than about 60% nucleotide identity, or about 65, 70, 75, 80, 85, or 95% nucleotide sequence identity, to a sequence of an SK2 gene, preferably a human SK2 gene as shown in SEQ ID NO: 2; or (4) encoded by a gene sequence that specifically hybridizes under stringent hybridization conditions to an SK2 gene, preferably a human SK2 gene as shown in SEQ ID NO: 2; or (5) encoded by a gene sequence that is amplifiable by primers that specifically hybridize under stringent hybridization conditions to an SK2 gene, preferably a human SK2 gene as shown in SEQ ID NO: 2.

Stringent hybridization conditions are well known in the art (see, for example, Maniatis et al., Molecular Cloning: A Laboratory Manual, Second Ed., Cold Spring Harbor Laboratory, Cold Spring Harbor, N.Y., 1989). Exemplary stringent hybridization conditions involve hybridization of a nucleic acid molecule on a filter support to a probe of interest at approximately 42° C. for about 8 to 24 hours in a low salt hybridization buffer, followed by washing at approximately 65° C. in a buffer comprising 0.02 to 0.04 M sodium phosphate, pH 7.2, 1% SDS and 1 mM EDTA for approximately 30 minutes to 4 hours. Conditions for increasing the stringency of a variety of nucleotide hybridizations are well known in the art.

An “SK2 gene” refers to a DNA molecule that: (1) encodes a protein having a sequence that has greater than about 60% amino acid identity, or about 65, 70, 75, 80, 85, 90, or 95% amino acid sequence identity, to a sequence of an SK2 protein, preferably a human SK2 protein as shown in SEQ ID NO: 4; or (2) encodes a protein capable of binding to antibodies (for example, polyclonal or monoclonal antibodies) raised against an immunogen comprising an SK2 protein, preferably a human SK2 protein as shown in SEQ ID NO: 4 or conservatively modified variants thereof; or (3) has greater than about 60% nucleotide identity, or about 65, 70, 75, 80, 85, or 95% nucleotide sequence identity, to a sequence of an SK2 gene, preferably a human SK2 gene as shown in SEQ ID NO: 2; or (4) specifically hybridizes under stringent hybridization conditions to an SK2 gene, preferably a human SK2 gene as shown in SEQ ID NO: 2; or (5) is amplifiable by primers that specifically hybridize under stringent hybridization conditions to an SK2 gene, preferably a human SK2 gene as shown in SEQ ID NO: 2.

With respect to proteins or peptides, the terms “isolated protein” or “isolated peptide” are sometimes used herein. This term may refer to a protein that has been sufficiently separated from other proteins with which it would naturally be associated, so as to exist in a “substantially pure” form. Alternatively, this term may refer to a protein produced by expression of an isolated nucleic acid molecule.

With reference to nucleic acid molecules, the term “isolated nucleic acid” is sometimes used. This term, when applied to DNA, refers to a DNA molecule that is separated from sequences with which it is immediately contiguous (in the 5′ and 3′ directions) in the naturally occurring genome of the organism from which it was derived. For example, the “isolated nucleic acid” may comprise a DNA molecule inserted into a vector, such as a plasmid or virus vector, or integrated into the genomic DNA of a prokaryote or eukaryote. An “isolated nucleic acid” molecule may also comprise a cDNA molecule. With respect to RNA molecules, the term “isolated nucleic acid” primarily refers to an RNA molecule encoded by an isolated DNA molecule as defined above. Alternatively, the term may refer to an RNA molecule that has been sufficiently separated from RNA molecules with which it would be associated in its natural state (for example, in cells or tissue), such that it exists in a “substantially pure” form.

Nucleic acid sequences and amino acid sequences can be compared using computer programs that align the similar sequences of the nucleic acids or amino acids, thus identifying the differences between the sequences. The BLAST programs (NCBI) and parameters used therein are used by many practitioners to align amino acid sequence fragments. However, equivalent alignments and similarity/identity assessments can be obtainable through the use of any standard alignment software (for example, ClustalW sequence alignment software).

As used herein, the term “cell” refers to at least one cell, and includes a plurality of cells appropriate for the sensitivity of the desired detection method. Cells suitable for use according to the invention can be prokaryotic or eukaryotic (for example, yeast, insect, mammalian, and the like). Preferred cells are mammalian cells.

The term “substantially the same” refers to nucleic acid or amino acid sequences having sequence variations that do not materially affect the nature of the corresponding protein, thus providing a functionally equivalent variant (for example, the structure, stability characteristics, substrate specificity and/or biological activity of the protein are not materially affected by the sequence variations). With particular reference to nucleic acid sequences, the term “substantially the same” is intended to refer to the coding region and to conserved sequences governing expression, and refers primarily to degenerate codons encoding the same amino acid, or alternate codons encoding conservative substitute amino acids in the encoded polypeptide. With reference to amino acid sequences, the term “substantially the same” refers generally to conservative substitutes and/or variations in regions of the polypeptide not involved in determination of structure or function of the protein.

The terms “percent identical” and “percent similar” are also used herein in comparisons among amino acid and nucleic acid sequences. When referring to amino acid sequences, “percent identical” refers to the percent of the amino acids of the subject amino acid sequence that have been matched to identical amino acids in the compared amino acid sequence by a sequence analysis program. “Percent similar” refers to the percent of the amino acids of the subject amino acid sequence that have been matched to identical or conserved amino acids. Conserved amino acids are those that differ in structure but are similar in physical properties such that the exchange of one for another would not appreciably change the tertiary structure of the resulting protein. Conservative substitutions are defined in Taylor (1986, J. Theor. Biol. 119:205). When referring to nucleic acid molecules, “percent identical” refers to the percent of the nucleotides of the subject nucleic acid sequence that have been matched to identical nucleotides by a sequence analysis program.

A “coding sequence” or “coding region” refers to a nucleic acid molecule having sequence information necessary to produce a gene product, when the gene is expressed.

Transcriptional and translational control sequences are DNA regulatory sequences, such as promoters, enhancers, polyadenylation signals, terminators, ribosome binding sites (for bacterial expression), operators, and the like, that provide for the expression of a coding sequence in a host cell. Regulatory sequences include those that direct constitutive expression of a nucleotide sequence in many types of host cells, as well as those that direct expression of a nucleotide sequence only in certain host cells (for example, tissue-specific regulatory sequences).

An “SK2 regulatory sequence” refers to a nucleic acid sequence that can control the expression of the SK2 gene or SK2 orthologs. The SK2 regulatory sequence includes a nucleic acid sequence having about 60% nucleotide sequence identity, preferably about 65, 70, 75, 80, 85, 90, or 95% nucleotide sequence identity, to nucleotides within the 2000 bp region immediately upstream (5′ direction) of the start codon for SK2. Characterization of the SK2 promoter region has been described, for example, by Kurima et al., J. Biol. Chem., 274:33306-33312 (1999).

According to some embodiments of the invention, an SK2 regulatory sequence can be operably linked to a gene. The gene under the control of the regulatory sequence can be any type of sequence that is detectable using the methods described herein. In some embodiments, useful genes can encode detectable markers such as proteins or enzymes as described herein (for example, reporter molecules). Alternatively, the gene under the control of the regulatory sequence can comprise a coding region of the SK2 gene. According to these embodiments, corresponding SK2 mRNA or SK2 protein can be detected using methods described herein.

Several assay methods can be used to measure the effect of a test molecule on the expression of a gene under control of the SK2 regulatory sequence. For example, gene or protein fusions comprising the SK2 regulatory sequence and a reporter gene can be used. The gene fusion is constructed such that only the transcription of the reporter gene is under control of the SK2 regulatory sequence. In some preferred embodiments, a second gene or protein fusion comprising the same reporter gene but a different regulatory sequence (for example, a regulatory sequence for a gene unrelated to the SK gene family) can be used as a control to increase the specificity of the assay. The effect of the test molecule on the expression of the reporter gene can be measured by methods known to those skilled in the art.

According to the invention, methods involving use of an SK2 regulatory sequence can not only identify molecules that regulate SK2 expression directly via binding to the SK2 regulatory sequence, but can also identify molecules that regulate SK2 expression indirectly via other mechanisms such as binding to other cellular components whose activities influence SK2 expression. For example, molecules that modulate the activity of a transcriptional activator or inhibitor for SK2 can be identified using the methods described herein.

The terms “promoter,” “promoter region,” or “promoter sequence” refer generally to transcriptional regulatory regions of a gene, which may be found at the 5′ or 3′ side of the coding region, within the coding region, and/or within introns. Typically, a promoter is a DNA regulatory region capable of binding RNA polymerase in a cell and initiating transcription of a downstream (3′ direction) coding sequence. The typical 5′ promoter sequence is bounded at its 3′ terminus by the transcription initiation site and extends upstream (5′ direction) to include the minimum number of bases or elements necessary to initiate transcription at levels detectable above background. Within the promoter sequence is a transcription initiation site (conveniently defined by mapping with nuclease S1), as well as protein binding domains (consensus sequences) responsible for the binding of RNA polymerase.

The term “operably linked” or “operably inserted” means that the regulatory sequences necessary for expression of the coding sequence are placed in a nucleic acid molecule in the appropriate position(s) relative to the coding sequence so as to enable expression of the coding sequence. This same definition is sometimes applied to the arrangement of other transcription control elements (for example, enhancers) in an expression vector.

As used herein, the term “reporter gene” refers to a nucleic acid sequence that encodes a reporter gene product. Correspondingly, the product encoded by the reporter gene (for example, mRNA or protein) is referred to as a “reporter molecule.” As is known in the art, reporter molecules are typically easily detectable by standard methods. Exemplary suitable reporter genes include, but are not limited to, genes encoding luciferase (lux), β-galactosidase (lacZ), green fluorescent protein (GFP), chloramphenicol acetyltransferase (CAT), β-glucuronidase, neomycin phosphotransferase, and guanine xanthine phosphoribosyl-transferase proteins.

A “vector” is a replicon, such as plasmid, phage, cosmid, or virus to which another nucleic acid segment may be operably inserted so as to bring about the replication or expression of the segment.

The term “selectable marker gene” refers to a gene encoding a product that, when expressed, confers selectable phenotype such as antibiotic resistance on a transformed cell.

As used herein, the terms “substantially purified” or “substantially pure” means that the protein or biologically active portion thereof is substantially free of cellular material or other contaminating proteins from the cell or tissue source from which the protein is derived, or substantially free of chemical precursors or other chemicals when chemically synthesized. The language “substantially free of cellular material” includes preparations of protein in which the protein is separated from cellular components of the cells from which it is isolated or recombinantly produced. Thus, protein that is substantially free of cellular material includes preparations of protein having less than about 30%, 20%, 10%, or 5% (by dry weight) of heterologous protein (also referred to herein as a “contaminating protein”). When the protein or biologically active portion thereof is recombinantly produced, it is preferably substantially free of culture medium, for example, culture medium represents less than about 20%, 10%, or 5 % of the volume of the protein preparation. When the protein is produced by chemical synthesis, it is preferably substantially free of chemical precursors or other chemicals, that is, it is separated from chemical precursors or other chemicals that are involved in the synthesis of the protein. Accordingly such preparations of the protein preferably have less than about 30%, 20%, 10%, 5% (by dry weight) of chemical precursors or compounds other than the polypeptide of interest. Purity is measured by methods appropriate for the compound of interest (for example, chromatographic methods, agarose or polyacrylamide gel electrophoresis, HPLC analysis, and the like).

A “control sample” or “control” refers to a sample that is compared with a test molecule, to identify molecules that affect expression of SK2 nucleic acid, expression of SK2 protein, or SK2 channel activity, utilizing such methods and assays as described herein. Typically, control samples can include a known amount of a test molecule, or a different molecule from the test molecule (for example, a molecule that is known to affect the aspect of the SK2 channel under observation, such as a known inhibitor or a known enhancer of SK2 channel activity or expression). In some embodiments, the control can be a value that is obtained utilizing the same method applied to analyze the test molecule, wherein the control value is obtained at the same time as analysis of the test molecule, or at a different time.

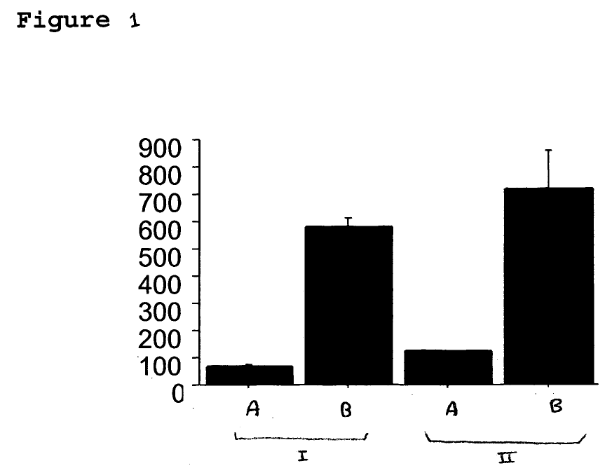

Turning to preferred embodiments of the invention, it has surprisingly been discovered that SK2 mRNA and protein levels in dorsal root ganglion (DRG) neurons are dramatically decreased in a neuropathic pain model compared to a control. As described in more detail in Example 1, and illustrated in FIG. 1, an established neuropathic pain model was utilized to observe decreased expression of SK2 mRNA and protein levels in mammalian DRG neurons. As one role of SK2 channels is to decrease neuronal excitability, the decreased levels of SK2 mRNA and protein in DRG can contribute to the development of the hyperexcitable state seen in the neuropathic pain animal models. In other words, decreased SK2 mRNA and/or protein expression can be correlated with neuropathic pain. Consequently, material that modulates SK2 mRNA and/or protein expression can be utilized to control neuropathic pain. In addition, assays for identifying molecules that modulate SK2 mRNA and/or protein expression are contemplated in the invention, as well as methods of treating neuropathic pain by administering compounds that increase SK2 mRNA or protein expression in a patient. Further, assays for identifying molecules that modulate activity of SK2 channels are contemplated, as well as methods of treating neuropathic pain by administering compounds that increase SK2 channel activity in a patient.

The preferred neuropathic pain model utilized herein was developed by Kim and Chung (Pain, 50:355-363 (1992)). In the model, both the L5 and L6 spinal nerves, or the L5 spinal nerve alone, on one side in rats were tightly ligated. The rats showed mechanical allodynia and thermal hyperalgesia of the affected hind paw lasting up to several months post-surgery. In addition, there was evidence of the presence of spontaneous pain. Therefore, this surgical paradigm produces behavioral signs in the rat that mimic some symptoms of neuropathic pain in humans. This model has been widely accepted as a neuropathic pain model and is referred to as the spinal nerve ligation (SNL) model of nerve injury.

In addition to the behavioral symptoms identified above, the SNL animals also exhibit cellular effects from the spinal nerve ligation. In the animal model, the transection or ligation of the spinal nerve results in immediate and irreversible interruption of electrical nerve conduction, followed by the appearance of sustained spontaneous electrical activity, Wallerian degeneration of axons distal to the lesion, and sprouting of the proximal axonal stumps in an attempt to regenerate the nerve fiber. Within days after the injury, chromatolysis of the nucleus is evident in the cell body in the DRG (Cragg, Brain Res. 23:1-21, 1970) and ectopic discharges are observed (Govrin-Lippmann R., Devor M., Brain Res. 159:406-10, 1978).

The present disclosure describes decreased expression of SK2 mRNA and protein in DRG in the SNL model compared to control. As described in Example 1, a significant decrease (more than five-fold) in expression of SK2 mRNA was observed in a neuropathic pain model. A significant decrease in SK2 protein expression was also observed via immunohistochemical analysis. This surprising discovery identifies a new therapeutic target for developing methods and strategies for treatment of neuropathic pain.

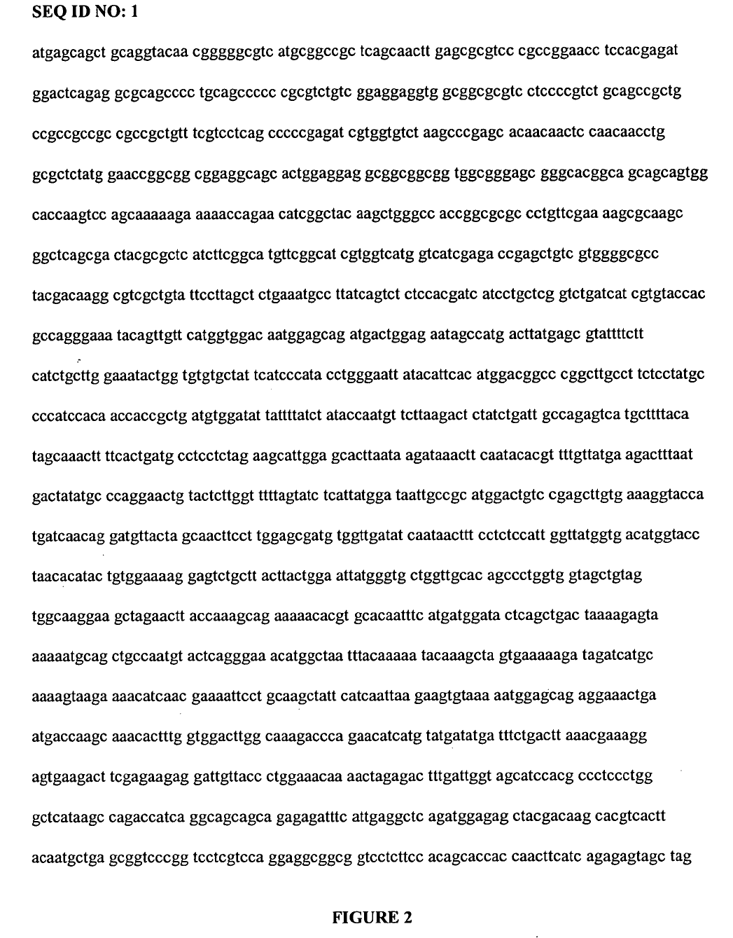

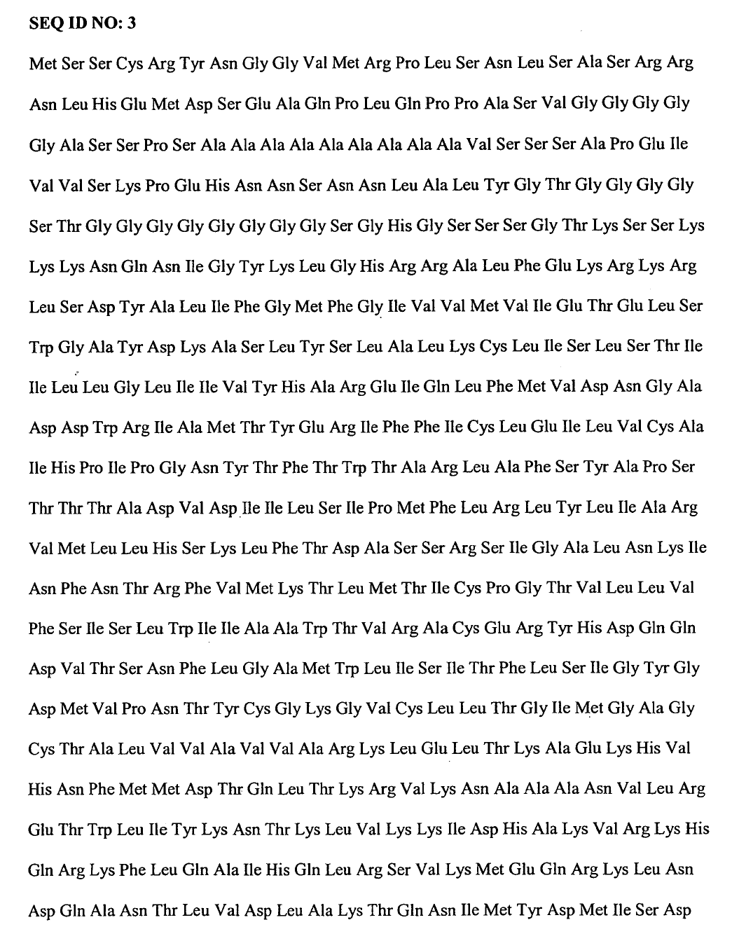

In another aspect, the present disclosure describes a novel human isoform of the SK2 gene, identified herein as hSK2A⁺ (SEQ ID NO: 1, FIG. 2). This novel isoform contains an in-frame insertion of 3 nucleotides at nucleotide position 173, coding for an alanine residue at position 58 of the corresponding amino acid sequence. Generally, alanine is an amino acid having an aliphatic side chain (methyl group) and carries an overall neutral charge.

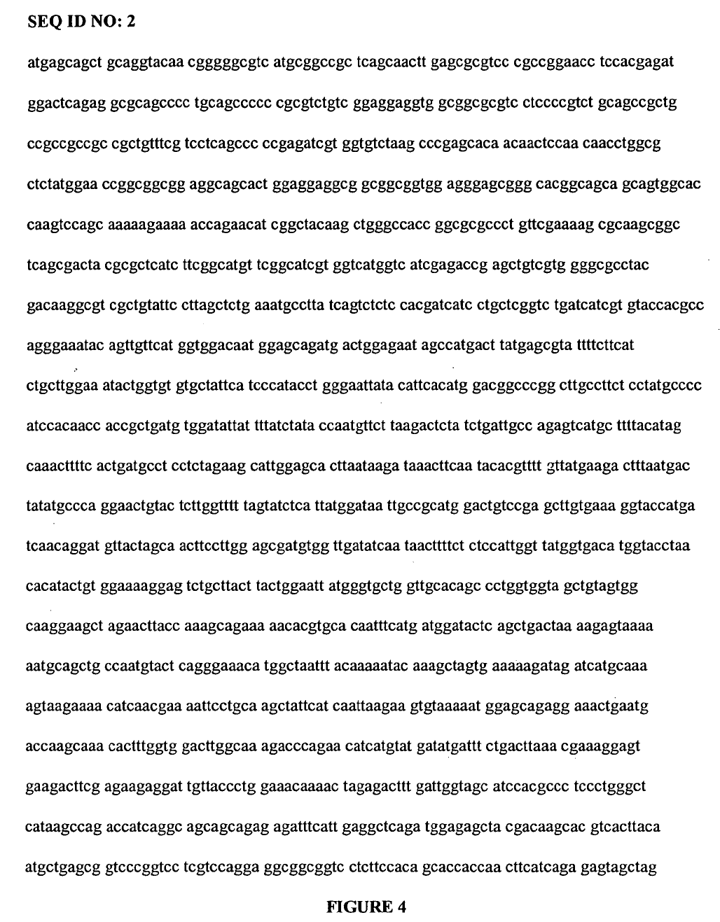

As described in more detail in the Examples, two SK2 clones were identified from human genome draft sequence using rat SK2 cDNA coding region as the query. The first clone, identified as hSK2A⁺ (SEQ ID NO: 1, FIG. 2) is discussed above. The second clone, identified as hSK2A⁻ (SEQ ID NO: 2, FIG. 4) was identical to the first clone except for the alanine insertion at nucleotide position 173.

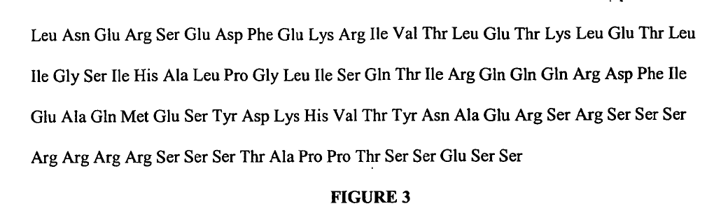

Alignment revealed that the coding sequence of SEQ ID NO: 1 (hSK2A⁺) is 99.4% identical to that of the hSK2 cloned from human leukemic Jurkat T cells (GenBank Access No. NM_{—}021614) and 91.7% identical to that of the rat rSK2 (GenBank Access No. U69882). The corresponding polypeptide sequence of hSK2A⁺ (SEQ ID NO: 3, FIG. 3) is 99.4% identical to the polypeptide encoded by hSK2 (GenBank Protein ID: NP 067627.1) and 97.4% identical to that encoded by rat rSK2 (GenBank Protein ID: AAB09563.1). SEQ ID NO: 2 (hSK2A⁻) is 99.9% identical to that of the hSK2 cloned from human leukemic Jurkat T cells (GenBank Access No. NM_{—}021614) and 92.0% identical to that of the rat rSK2 (GenBank Access No. U69882). With respect to SEQ ID NO: 2 comparison with hSK2, the differences in nucleic acid sequences were accounted for by conservative substitutions (substitutions that did not affect the corresponding amino acid sequence). The corresponding polypeptide sequence of clone hSK2A⁻ (SEQ ID NO: 4) is 99.9% identical to the polypeptide encoded by hSK2 (GenBank Protein ID: NP 067627.1) and 97.6% identical to that encoded by rSK2 (GenBank Access No. U69882).

Each of the protein products of the two clones were independently expressed in an oocyte expression system (Example 4) and a mammalian expression system (Example 5). In each case, the SK2 protein products of the clones formed functional calcium-activated potassium channels. Moreover, the SK channels were activated by known SK2 activators (chlorzoxazone) and produced similar whole cell currents to those reported for known SK2 channels. The SK channels formed by the expressed SK2 proteins were also blocked by a known SK2 inhibitor (apamin), and the reversal potential for the SK2 currents was as predicted for a potassium current mediated by SK2 channels. Results showed that the two hSK2 clones identified herein could be expressed, and the SK2 proteins were capable of forming functional SK2 channels, in each expression system. The additional alanine did not appear to affect the ability of the novel hSK2A⁺ isoform to form a functional channel in either expression system.

Regarding the novel SK2 isoform identified herein, hSK2A⁺-encoding nucleic acids can be used for a variety of purposes in accordance with the present invention. The DNA, RNA, or fragments of the DNA or RNA of the novel hSK2A⁺ isoform can be used as probes to detect the presence and/or expression (transcription or translation) of SK2 in a system. Methods in which hSK2A⁺-encoding nucleic acids can be utilized as probes for such assays include, but are not limited to, in situ hybridization, Southern hybridization, Northern hybridization, and assorted amplification reactions such as polymerase chain reactions (PCR).

hSK2A⁺-encoding nucleic acids of the invention can also be utilized as probes to identify related genes from other species. As is well known in the art, hybridization stringencies can be adjusted to allow hybridization of nucleic acid probes with complementary sequences of varying degrees of homology.

In further embodiments, the novel isoform can be utilized to create transgenic cells, tissues or organisms. For example, hSK2A⁺ can be used to increase SK2 expression in a cell, for example, to treat neuropathic pain. In yet further embodiments, the novel hSK2A⁺ isoform can be utilized to increase SK2 activity in a cell, for example, to treat neuropathic pain.

In some cases, the novel isoform hSK2A⁺ may be used as a marker to identify subpopulations of individuals, as it is known that certain polymorphisms are associated with particular phenotypic traits.

The hSK2A⁺ isoform includes not only the identified insertion isoform, but also such variants as addition, deletion, and/or substitution isoforms, as well as fragments of the isoform.

In another aspect, the invention provides assay systems and methods for identifying molecules that (1) affect the functional expression of an SK2 protein; (2) affect the function or activity of an SK2 channel (such as, for example, the open probability of an SK channel, or the ionic conductance of an SK channel); and/or (3) bind the SK2 channel or a protein subunit of the SK2 channel.

According to the invention, test molecules are subjected to the inventive assays to determine the affect the test molecule has on expression of SK2 nucleic acid, SK2 protein, and/or activity of an SK2 channel. Thus, as used herein, a “test” molecule is a molecule suspected to affect one of the characteristics of the SK2 channel as described herein. In preferred embodiments, the inventive assay systems and methods are used to identify modulators of SK2 channels. As used herein, SK2 channel “modulators” include molecules that interact with an SK2 channel in such a way as to affect the activity and/or expression of the SK2 channel. Illustrative examples of modulators include molecules that decrease, block, prevent, delay activation, inactivate, desensitize or down regulate channel activity, or speed or enhance deactivation of the SK2 channel, such as, for example, channel inhibitors or blockers. Other illustrative examples of modulators include molecules that increase, open, activate, facilitate, enhance activation, sensitize or up regulate channel activity, or delay or slow inactivation of the channel, such as, for example, channel activators or openers.

In preferred embodiments, the inventive assay systems and methods are utilized to identify molecules that increase activity and/or expression of SK2 channels to a level that treats and/or alleviates neuropathic pain. These and other preferred embodiments will now be described in more detail.

In one aspect, the invention provides a method comprising steps of (a) providing cells capable of expressing SK2; (b) contacting the cells with a test molecule; (c) obtaining information indicative of cellular SK2 expression to obtain an SK2 Expression Value; (d) comparing the SK2 Expression Value with a control SK2 Expression Value; and (e) identifying a test molecule that causes the cells to display an SK2 Expression Value that is different from the control SK2 Expression Value. In preferred embodiments, the method involves identifying a test molecule that causes the cells to display an SK2 Expression Value that is greater than the control SK2 Expression Value.

In some embodiments, the step of obtaining information indicative of cellular SK2 expression comprises analyzing SK2 mRNA expression. In other embodiments, the step of obtaining information indicative of cellular SK2 expression comprises analyzing SK2 protein expression. Alternatively, the step of obtaining information indicative of cellular SK2 expression comprises analyzing expression of a gene under the control of an SK2 regulatory sequence, for example, a reporter molecule.

The inventive method provides an SK2 Expression Value, which is compared with a control SK2 Expression Value. The SK2 Expression Value is any quantitative aspect of SK2 expression or a gene under the control of an SK2 regulatory sequence, as described herein. In one embodiment, the control SK2 Expression Value is obtained from cells that are not in contact with the test molecule. In another embodiment, the control SK2 Expression Value is obtained from cells that are in contact with a molecule known to affect SK2 expression, such as, for example, a known inhibitor or enhancer of SK2. According to these embodiments, the molecule known to affect SK2 expression is provided in a known amount to the cells of the control.

For example, the effect of the test molecule on expression of SK2 channels can be determined by assigning a relative SK2 Expression Value of 100% (in the case of an activator or enhancer of SK2 channel expression) or 0% (in the case of a known inhibitor of SK2 channel expression), and observing a change in the Expression Value relative to the control. In some embodiments, activation of SK2 channels can be determined by assigning a relative SK2 Expression Value of 100% to control samples (the “control Expression Value”) and observing an increase in SK2 expression relative to the control Expression Value. Preferably, the methods of the invention are utilized to identify a test molecule that causes the cells to display an SK2 Expression Value of at least 200% relative to the control, or at least 300%, preferably at least 500% relative to the control. In some embodiments, inhibition of SK2 channels can be determined by observing a decrease in SK2 Expression Value relative to the control Expression Value, for example, when the SK2 Expression Value relative to control is about 10% or less, preferably about 20% or less relative to the control.

When the method involves a gene under the control of an SK2 regulatory sequence, a gene Expression Value is obtained, which is compared with a control gene Expression Value. The preceding discussion of the Expression Value is applied to these embodiments as well.

According to the invention, any cell type that is capable of expressing functional SK2 can be used, such as, for example, naturally occurring, artificial, or modified cells. Naturally occurring cells include cells that naturally express SK2 without manipulation of a genetic or biochemical feature of the cell to achieve or affect such SK2 expression. Examples of naturally occurring cell types include, but are not limited to, DRG, nodose, trigeminal, proximal colon, cells in numerous brain regions including neocortex, hippocampus, reticularis thalami, and inferior olivary nucleus, dentate gyrus, olfactory bulb and anterior olfactory nucleus, cerebellum, pontine nucleus, and adrenal gland, and non-excitable cells including but not limited to lymphocytes. Mammalian cells are preferred, particularly neural cells, in particular dorsal root ganglion cells. Procedures for isolating these cells from their respective tissues are known in the art.

“Modified cells” refers to cells that have been manipulated (by man or by nature) in a way to change a certain genetic or biochemical feature of the cell. For example, modified cells include transfected cells, transgenic cells, and hybridomas. In some embodiments, cells can be transfected with a nucleic acid that is capable of expressing SK2, as described herein. The SK2 can be expressed, for example, from a vector that is either stably or transiently transfected into the cell. Vectors suitable for SK2 expression are known in the art, particularly vectors allowing SK2 expression in a mammalian cell, and commercially available from, for example, Promega. Examplary modified cells are found in the Examples, where hSK2(A⁺)/tsA201 cells and hSK2(A−)/tsA201 cells were created.

In some cases it can be desirable to express a variant of an SK2 channel in a cell. For example, variants may reveal higher or lower activity than wildtype channels, may act as dominant negative suppressors of native SK2 function and/or may be useful as a gene therapy. Cells can also be transfected with a nucleic acid having a gene under the control of an SK2 regulatory sequence. According to preferred embodiments of the invention, the SK2 regulatory sequence is sufficient to drive expression of the gene in response to an SK2 activating molecule. In some cases, a nucleic acid having most or all of the regulatory region of SK2 (preferably at least a portion of the 2000 bp region immediately upstream of the start codon for SK2, as described herein) operably linked to a gene can be prepared and introduced into a cell in a vector. Artificial cells include manufactured cells, for example, membrane encapsulated vesicles.

In another aspect, cells according to the invention can express endogenous SK2 nucleic acid, or exogenous SK2 nucleic acid. Cells that express endogenous SK2 nucleic acid can include naturally-occurring cells, or modified cells. The SK2 nucleic acid can be obtained from human or other suitable mammalian species. Examples of modified cells that express endogenous SK2 can include cells modified to include a promoter or enhancer of SK2 expression. Examples of cells that express endogenous SK2 include such human cells as primary human hepatocytes, human HuH-7 hepatoma cells, and human Mz-ChA-1 cholangiocarcinoma cells (see Roman et al., American J. of Physiol., 282(1)G116-G122 (2002)); human leukemic Jurkat T cells (Desai et al., J. of Biol. Chem., 275(51):39954-39963 (2000)); as well as murine cells such as mouse osteocyte-like cell line MLO-Y4 (Gu et al., Bone, 28(1):29-37 (2001)). Examples of modified cells that express exogenous SK2 include Chinese hamster ovary (CHO-K1) cells transfected to express SK2, as described in Dale, et al., Naunyn-Schniedeberg's Archives of Pharmacology, 366(5), 470-477 (2002).

According to the invention, the method utilizes cells capable of expressing SK2. Such cells can express SK2 at any desirable level. For example, when it is desirable to identify a modulator that increases SK2 expression, it can be desirable to utilize SNL cells, since such cells would provide low levels of SK2 expression, and an increase in SK2 expression upon exposure to a test molecule could be readily observed (whereas a decrease in SK2 expression may be more difficult to observe). In another exemplary embodiment, when it is desirable to identify a modulator that decreases SK2 expression, it can be desirable to utilize cells that provide higher levels of SK2 expression, such that a decrease in SK2 expression upon exposure to a test molecule could be readily observed. The level of SK2 expression in the cells of the invention can be chosen in accordance with the principles described in this disclosure. Preferably, human cells are utilized in assays of the invention. Optionally, cells obtained from other species, such as rat, mouse, or other suitable systems, preferably a mammalian species, are used in accordance with the invention.

As mentioned, some embodiments of the invention involve an SK2 regulatory sequence. According to these particular embodiments, the invention provides methods comprising steps of: (a) providing a sample capable of expressing SK2; (b) contacting the sample with a test molecule; (c) obtaining information indicative of SK2 expression in the sample to obtain an SK2 Expression Value; (d) comparing the SK2 Expression Value with a control SK2 Expression Value; and (e) identifying a test molecule that causes the sample to display an SK2 Expression Value that is different from the control SK2 Expression Value. In preferred embodiments, the sample can comprise an in vitro system, wheat germ extract, or reticulocyte extract. The SK2 regulatory sequence can be utilized in a cell-based assay, or a cell-free assay. Examples of suitable cell-free assay systems include in vitro translation and/or transcription systems, which are known to those skilled in the art. For example, full length SK2 cDNA, including the regulatory sequence, can be cloned into an expression vector. Using this construct as the template, SK2 protein can be produced in an in vitro transcription and translation system. Alternatively, synthetic SK2 mRNA or mRNA isolated from SK2 protein producing cells can be efficiently translated in various cell-free systems, including but not limited to wheat germ extracts and reticulocyte extracts. The effect of the test molecule on the expression of the SK2 gene or reporter gene controlled by the SK2 regulatory sequence can be monitored by direct measurement of the quantity of SK2 mRNA or protein, or reporter molecule (mRNA or protein) in the reaction solution using methods described herein.

Cells capable of expressing SK2 are contacted with a test molecule. In some embodiments, the amount of time required for contact with the test molecule can be empirically determined by running a time course with a known SK2 modulator, such as apamin, and measuring cellular changes as a function of time.

Once cells or samples are contacted with a test molecule, information indicative of expression of SK2 is obtained, to obtain an SK2 Expression Value. Expression of SK2 in the cells or sample can preferably be determined by detection of SK2 mRNA, SK2 protein, and/or level of reporter molecule in the cells or sample. Generally, test molecules can affect the SK2 Expression Value relative to control by affecting SK2 gene transcription and/or translation.

The presence and/or amount of SK2 mRNA in a sample can be detected using a variety of techniques. Generally speaking, SK2 mRNA can be analyzed utilizing in situ hybridization techniques; isolation of mRNA, followed by detection and/or measurement; polymerase chain reaction (PCR) and variations of the PCR; and/or microarray techniques.

In one embodiment, SK2 mRNA is analyzed by in situ hybridization. For example, SK2 mRNA can be detected and/or measured by contacting the sample with a compound or an agent capable of specifically detecting the SK2 mRNA. In one preferred embodiment, SK2 mRNA can be contacted with a labeled nucleic acid probe capable of hybridizing specifically to the SK2 mRNA. Preferably, the nucleic acid probe specific for SK2 mRNA is a full-length human SK2 cDNA as described herein, or a portion thereof, such as an oligonucleotide of at least 15, 30, 50, 100, 250, or 500 nucleotides in length of human SK2 mRNA, and sufficient to hybridize to an SK2 mRNA under stringent conditions. Other suitable probes can be substituted, for example, hSK2A⁺, SK2 cDNA from other species, and the like. Preferably, a nucleic acid probe specific for SK2 mRNA will only hybridize to SK2 mRNA under stringent conditions, not to other nucleic acids present in the assayed sample. The labeled probe can be radioactive or enzymatically labeled. One suitable method of in situ hybridization is described in Example 1, and other suitable methods known in the art can be substituted for the described method.

Alternatively, analysis of SK2 mRNA can involve isolation of the mRNA, followed by detection and/or measurement. For example, SK2 mRNA can be isolated and analyzed by the Northern blot method. In one exemplary embodiment, mRNA is isolated by the acid guanidinim thicyanatephenol:chloroform extraction method (Chomczynski et al., Anal. Biochem., 162:156-159 (1987)) from cell lines or tissues of a subject. Extracted mRNA can then be separated by gel electrophoresis under denaturing conditions, then transferred to a nylon membrane, where the mRNA is detected by hybridization to a labeled probe. Alternatively, the format of the blotting can be altered from transfer from a gel to direct application to slots on a specific blotting apparatus containing the nylon membrane (slot or dot blotting), which eliminates the need for gel electrophoresis.

Other useful techniques for analyzing SK2 mRNA in a sample utilize the polymerase chain reaction (PCR) and variations of the PCR. For example, quantitative PCR can be utilized to determine the level of mRNA production. Such methods can involve the comparison of a standard or control DNA template amplified with separate primers at the same time as the specific target DNA. Other methods involve the incorporation of a radiolabel through the primers or nucleotides and their subsequent detection following purification of PCR product. An alternative method is the 5′-exonuclease detection system (Taqman™, Roche Molecular Systems, Inc.) assay. According to this method, an oligonucleotide probe is labeled with a fluorescent reporter and quencher molecule at each end. When the primers bind to their target sequence, the 5′-exonuclease activity of Taq polymerase degrades and releases the reporter from the quencher. A signal is thus generated that increases in direct proportion to the number of starting molecules. Thus, the detection system is able to induce and detect fluorescence in real time as the PCR proceeds. Another useful technique for determining the presence and/or amount of SK2 mRNA in a sample includes performing reverse transcriptase-polymerase chain reaction (RT-PCR). According to this embodiment, cDNA can be prepared from a sample treated with the test molecule and SK2 cDNA amplified using oligonucleotide primers specific for the SK2 sequence and able to hybridize to the SK2 cDNA under stringent PCR conditions. Kits are commercially available that facilitate the detection of PCR products that incorporate detectable labels, for example SYBR™ Green PCR Core Reagents (Applied Biosystems, Foster City, Calif.).

Other useful techniques for analyzing SK2 mRNA in a sample include DNA microarray techniques, which are common in the art and will not be described in further detail herein.

The presence and/or amount of SK2 protein in a sample can be analyzed by contacting the sample with a compound or an agent capable of specifically detecting the SK2 protein. Analysis of SK2 protein can be performed in situ, or SK2 protein can first be isolated prior to analysis. A preferred agent for detecting SK2 protein is an antibody capable of binding specifically to a portion of the SK2 polypeptide. In one preferred method, an antibody specific for SK2 coupled to a detectable label is used for the detection of SK2. Antibodies specific for SK2 can be polyclonal or monoclonal. A whole antibody molecule or a fragment thereof (for example, Fab or F(ab′)₂) can be used.

Other techniques for detecting SK2 protein include enzyme linked immunosorbent assays (ELISAs), Western blots, immunoprecipitation, and immunofluorescence. All these methods are known to those skill in the art.

One illustrative immunocytochemical method is described in Example 1. Other known immunocytochemical methods can be utilized in accordance with the invention. Methods for visualization of the label of the antibody commonly include fluorescence, enzymic reactions, and gold with silver enhancement. For example, Western blotting involves isolation of protein from the sample, followed by separation on polyacrylamide gel. The separated proteins are then transferred from the gel to a nitrocellulose paper. The blot is then probed, usually using an antibody to detect SK2. The blot is first incubated in a protein solution, for example, 10% (w/v) BSA, or 5% (w/v) non-fat dried milk, which will block all remaining hydrophobic binding sites on the nitrocellulose sheet. The blot is then incubated in a dilution of primary antibody directed against SK2, then secondary antibody that is appropriately labeled for visualization on the blot. The label for the secondary antibody can be an enzyme (such as, for example, alkaline phosphatase or horseradish peroxidase), a radioisotope (for example, ¹²⁵I), a fluorescein isothiocyanate label, gold label, or biotin. Other suitable labels are known in the art and can be substituted for those specifically identified herein.

Assays to detect SK2 protein can also be performed with purified SK2 protein or microsomes containing SK2 proteins derived from native tissue or cell lines (Schetz J A, (1995), Cardiovascular Research; 30:755-762).

In some embodiments, obtaining information indicative of SK2 expression can comprise determining the presence of a reporter molecule in the cells. In some preferred embodiments, the reporter gene is coupled with an SK2 regulatory sequence, as described in more detail elsewhere herein. One of skill in the art would readily appreciate that reporter mRNA and/or protein can be detected in order to obtain information indicative of expression of SK2, in accordance with the invention.

In one embodiment, the invention provides a method comprising steps of (a) providing a sample comprising an SK2 channel; (b) contacting the sample with a test molecule; (c) obtaining information indicative of SK2 channel activity in the sample to obtain an SK2 Channel Activity Value; (d) comparing the SK2 Channel Activity Value with a control Channel Activity Value; and (e) identifying a test molecule that causes the SK2 Channel Activity Value to be different from the control Channel Activity Value. Preferably, the identifying step comprises identifying a test molecule that causes the SK2 Channel Activity Value to be greater than the control Channel Activity Value. In preferred embodiments, the identifying step comprises identifying a test molecule that causes the SK2 Channel Activity Value to be at least 120% of the control Channel Activity Value, preferably at least 150% of the control Channel Activity Value.

According to this embodiment, the inventive method involves obtaining an SK2 Channel Activity Value, which is compared to a control SK2 Channel Activity Value. The Channel Activity Value is any quantitative aspect of SK2 channel activity, as described herein. In one embodiment, the control SK2 Channel Activity Value is obtained from cells that are not in contact with the test molecule. Activation of SK2 channels can be determined by assigning a relative SK2 Channel Activity Value of 100% to control samples (the “control Channel Activity Value”) and observing an increase in SK2 channel activity relative to the control Channel Activity Value. Preferably, the methods of the invention are utilized to identify molecules that cause the SK2 Channel Activity Value to be at least about 110%, or 150%, or 200% of the control Channel Activity Value, or at least 300% of the control Channel Activity Value, preferably at least 500% of the control Channel Activity Value, more preferably at least 1000% of the control Channel Activity Value. Inhibition of SK2 channels is achieved when the SK2 Channel Activity Value relative to control is about 90% or less, or 75% or less, or 50% or less, preferably in the range of about 25% to about 0%.

In another embodiment, the control SK2 Channel Activity Value is obtained from cells that are in contact with a molecule known to affect SK2 channel activity, such as, for example, a known inhibitor or enhancer of SK2. According to these embodiments, the molecule known to affect SK2 channel activity can be provided in a known amount to the cells of the control. The effect of the test molecule on SK2 channel activity can be determined by assigning a relative SK2 Channel Activity Value of 100% (in the case of an activator or enhancer of SK2 channel activity) or 0% (in the case of a known inhibitor of SK2 channel activity), and observing a change in the Channel Activity Value relative to the control. The extent to which activation of SK subunits results in significant depression of cell electrical activity will depend upon cellular parameters such as membrane conductance and/or membrane potential, such that in some cases the activation of only a small percentage of SK2 channels can significantly alter cellular physiology.

According to this embodiment of the invention, test molecules suspected to affect the activity of an SK2 channel are contacted with biologically active SK2 channels, either recombinant or naturally occurring. SK2 channels can be isolated in vitro, expressed in a cell, or expressed in a membrane derived from a cell. In such assays, an SK2 polypeptide is expressed to form an SK2 channel.

Preferably, the SK2 channels are provided in a cell membrane. Cell membranes can be obtained from any suitable type of animal cell, including human, rat, and the like. Whole cells can be isolated and treated using methods known in the art for cell preparation, including mechanical or enzymic disruption of the whole tissue, or by cell culture. In some embodiments, it can be preferable to utilize whole cells as the source of cell membrane, for example, when the cell membrane preparation procedure can destroy or inactivate cell receptors.

In some preferred embodiments, membranes can be broken under controlled conditions, yielding portions of cell membranes and/or membrane vesicles. Cell membrane portions and/or vesicles can, in some embodiments, provide an easier format for the inventive assays and methods, since cell lysis and/or shear is not as much of a concern during the assay. Cell membranes can be derived from tissues and/or cultured cells. Such methods of breaking cell membranes and stabilizing them are known in the art. Methods of treating tissues to obtain cell membranes are known in the art.

The SK2 channels contained within the cell membrane can be obtained from naturally-occurring, artificial, or modified cells, as described elsewhere herein. Further, as described elsewhere herein, the SK2 channels can be formed from cells that express endogenous SK2 nucleic acid or exogenous SK2 nucleic acid. As described in the Examples, hSK2 has been successfully expressed in Xenopus oocytes (Example 4) and tsA201 cells (Example 5).

In another embodiment, SK2 channels can be incorporated into artificial membranes (see Cornell, B A, et al., Nature 387,580-583 (1997)). For example, such artificial membranes can include an electrode to which is tethered a lipid membrane containing ion channels and forming ion reservoirs.

Preferably, human SK2 channels are used in the assays of the invention. Optionally, SK2 orthologs from other species such as rat or mouse, preferably a mammalian species, are used in assays of the invention.

In some embodiments, for example, when a biologically active SK2 channel is not required, the inventive methods can utilize an SK2 protein that comprises a subunit of an SK2 channel. As described earlier, an SK2 channel is composed of SK2 protein subunits that assemble and complex with calmodulin, thereby forming an active SK2 channel. Thus, in embodiments where binding of a test molecule to an SK2 protein is being determined, and activity of the channel is not relevant to the assay, the methods of the invention can utilize SK2 protein that is not necessarily assembled into an active SK2 channel. Such assays, in some embodiments, can be cell-free assays, as described in more detail elsewhere herein. For example, one or more SK2 protein subunits of the channel can be incorporated into lipid bilayer, with or without calmodulin.

In addition, ion channels can be functionally expressed in lipid bilayers using established methods, and their activity can be manipulated by changes in membrane potential across the lipid and addition of Ca²⁺ and other necessary components to the chambers. Since the SK2 complex requires calmodulin to be functional, calmodulin is included. Changes in single channel conductance can be measured readily with this technique when few channels are incorporated. In alternative embodiments, use of a channel that does not include calmodulin can be useful in identifying activators of channel in the absence of a calcium-dependent activation mechanism. The calmodulin binding sites can be altered to create an SK2 channel that can be used to screen for activators independent of intracellular Ca²⁺levels.

According to the invention, sample comprising an SK2 channel is contacted with a test molecule. In some embodiments, the amount of time required for contact with the test molecule can be empirically determined by running a time course with a known SK2 modulator, such as apamin, and measuring cellular changes as a function of time.

In preferred embodiments, the inventive assay systems and methods are utilized to identify molecules that increase activity of SK2 channels to a level that treats and/or alleviates neuropathic pain. In some preferred embodiments, the molecule increases the SK2 Channel Activity Value to at least 150% relative to a control.

Generally, SK2 channel activity is analyzed by measuring ionic conduction across a biological membrane. SK2 channel activity can be analyzed utilizing a variety of techniques. Ionic conduction can be measured directly by such methods as current measurement (for example, patch-clamping) and radioactive and non-radioactive ion flux assays which quantify the change of the concentration of the conducted ions. Indirect assays include fluorescent voltage-sensitive probes which measure membrane potential changes caused by ion flux as long as the membrane potential is different from the equilibrium potential for the ion. In addition to the above functional assays, binding assays can be utilized to study ion channel targets. Additionally, pain responses in animal models can be studied to analyze behavioral responses. Exemplary embodiments of methods of analyzing channel activity are described in more detail below.

Ionic conduction can be measured directly by such methods as current measurement and radioactive or non-radioactive ion flux assays. Current through the SK2 channel can be measured utilizing any suitable technique. A preferred method to determine changes in cellular polarization is by measuring changes in current (thereby measuring changes in polarization) with voltage-clamp and patch-clamp techniques, for example, the “cell-attached” mode, the “inside-out” mode, and the “whole cell” mode (see, for example, Ackerman et al., New Engl. J. Med. 336:1575-1595 (1997)). Whole cell currents are conveniently determined using the standard methodology (see, for example, Hamil et al., Pflugers. Archiv. 391:85 (1981)).

Patch-clamp techniques generally involve a glass micropipette with a tip diameter on the order of micrometers, which is brought in contact with the membrane of a cell. The glass forms a high resistance seal with the membrane, thus electrically isolating the patch of membrane covered by the pipette tip. Patch clamping can also be done using other substrates such as planar electrodes. The salt solution in the electrode is connected through an Ag/AgCl junction to a device that allows simultaneous recording of current and control of potential (“voltage clamp”) or measure the voltage with or without the injection of current to change the potential (“current clamp”) over the patch of membrane. If the patch contains one or only a few ion channels, ionic currents through individual channels can be recorded. In some embodiments, patch clamp studies can offer several advantages, such as flexibility in manipulating the intra- or extracellular medium during the experiment, and the ability to record single-channel activity and/or whole-cell (macro)currents.

In one embodiment of the invention, spontaneous discharges from neurons (ectopic activity) can be measured ex vivo, as described in more detail in Example 2. Briefly, the spinal nerves under investigation are removed with attached dorsal root ganglia. The neurons are then placed in an in vitro recording chamber that consists of two chambers, one for the dorsal root and the other for the dorsal root ganglia and spinal nerve. The DRG and spinal nerve compartment is perfused with oxygenated artificial cerebrospinal fluid, and the dorsal root compartment is filled with mineral oil. The spinal nerve is stimulated using a suction electrode, and spontaneous discharges are recorded from the teased dorsal root fascicles. This method allows the effects of various compounds on ectopic activity to be readily determined by adding the test molecule and measuring the number of spontaneous discharges observed over time, compared to control.

The effects of the test molecules upon the activity of the channels can be measured by changes in the electrical currents or ionic flux, or by the consequences in changes in currents and flux. Changes in the electrical current or ionic flux can be measured by either an increase or decrease in flux of ions such as potassium, rubidium, or cesium ions. The cations can be measured in a variety of standard ways. They can be measured directly by concentration changes of the ions, or indirectly by membrane potential, by radiolabeling of the ions, or by using atomic adsorption spectroscopy methods to measure the concentration of non-radioactive ions. Consequences of the test molecule on ion flux can be quite varied. Accordingly, any suitable physiological change can be used to assess the influence of a test molecule on the SK2 channels. In some embodiments, for example, the effects of a test molecule can be measured by a toxin binding assay. When the functional consequences are determined using intact cells or animals, one can also measure a variety of effects such as transmitter release, hormone release (for example, insulin), transcriptional changes to both known and uncharacterized genetic markers (for example, utilizing Northern blots), and changes in intracellular second messengers such as Ca²⁺ or cyclic nucleotides.

In some embodiments, assays can include radiolabeled rubidium flux assays and fluorescence assays using voltage sensitive dyes (see, for example, Vestergarrd-Bogind et al., J. Membrane Biol. 88:67-75 (1988); Daniel et al., J. Pharmacol. Meth. 25:185-193 (1991); Holevinsky et al., J. Membrane Biology 137:59-70 (1994)). Assays for compounds capable of inhibiting or increasing potassium flux through the SK2 channel can be performed by application of the compounds to a bath solution in contact with and comprising cells having a channel of the present invention (see, for example, Blatz et al., Nature 323:718-720 (1986); Park, J. Physiol. 481:555-570 (1994)). Generally, the compounds to be tested are present in the range of about 0.0001 mM to about 0.3 mM.

The technique of atomic adsorption spectroscopy can be used to determine the flux of a number of ions including Rb⁺ and Cs⁺. Since Cs⁺ permeates the SK channel, cells can be exposed to extracellular Cs⁺ in the presence of test molecules. Channel openers will increase intracellular Cs⁺ levels.

Compounds that increase the flux of ions can cause a detectable increase in the ion current density by increasing the probability of an SK2 channel being open (the “open probability” of the SK2 channel), by decreasing the probability of it being closed, by increasing conductance through the channel, and/or by allowing the passage of ions.

In some embodiments, SK2 channel activity is determined by analysis of membrane potential (the potential difference across the cell membrane). Although cells (such as neural cells, for example) contain an equal number of anions and cations and are electrically neutral, the concentration of individual ions is often grossly different within the cell relative to the external environment. As discussed herein, neural cells couple the concentration of Ca²⁺ with action potential firing. Owing to differences in the permeability of the cell membrane to different ions, most cells possess a membrane potential such that the inside of the cell is negative relative to the outside. The membrane potential in resting neurons is typically approximately −70 mV. When the depolarization reaches approximately −55 mV (the threshold for these cells) a neuron will fire an action potential. The precise value of the membrane potential is dictated by the Nernst equation. For measurement of the membrane potential, a microelectrode connected to an electronic amplifier is typically inserted through the membrane into the cell. The measured constant negative potential difference is the resting potential. Vₘ=membrane potential.

Preferably, analysis of membrane potential involves membrane potential sensitive dyes, such as membrane potential sensitive fluorescent dyes. Suitable membrane potential sensitive fluorescent dyes are commercially available and have been employed in studies of cell physiology, particularly neurophysiology. Types of dyes available and the technologies of utilizing such dyes to measure membrane potentials are known to those skilled in the art. One example of a fluorescence assay utilizing a membrane potential sensitive dye is described in Example 6.

Information indicative of SK2 channel activity can be obtained utilizing any suitable control, as described elsewhere herein. In addition, in some embodiments, information indicative of the Channel Activity Value can be obtained by comparing two calmodulin-expressing cells, one containing an SK2 channel subunit and a second cell identical to the first, but lacking the SK2 channel subunit. After both cells are contacted with the same test molecule, differences in SK2 activities between the two cells are compared. This technique is also useful in establishing the background noise of assays. Background can also be obtained using an inhibitory compound at a concentration that blocks the channel activity. One of ordinary skill in the art will appreciate that these control mechanisms also allow easy selection of cellular changes that are responsive to modulation of functional SK2 channels.

In some embodiments, the invention involves binding assays to identify test molecules that are potentially capable of affecting activity and/or expression of an SK2 channel. Particularly useful binding assays are competitive binding assays, which preferably involve the use of labeled ligand that specifically binds SK2 channel, SK2 mRNA, and/or SK2 protein. Compounds identified in these binding assays can, in some preferred embodiments, be further characterized by subjecting the compounds to methods for determining their effect on SK2 expression, SK2 channel activity, behavior tests to identify phenotypic characteristics in animals that are exposed to the compounds, and/or other assays as described herein.

In one such embodiment, the invention provides a method for identifying a molecule that binds an SK2 channel and is potentially capable of affecting expression or function of the SK2 channel. One embodiment of the method comprises (a) providing sample containing an SK2 channel; (b) incubating the sample with a labeled ligand selected to specifically bind the SK2 channel, under conditions sufficient to allow the labeled ligand to bind the SK2 channel; (c) incubating the sample with a test molecule; (d) separating unbound labeled ligand from SK2 channel; (e) detecting binding of labeled ligand to the SK2 channel, wherein a change in the binding of the labeled ligand to the SK2 channel in the presence of the test molecule as compared to the absence of the test molecule, indicates that the test molecule is potentially capable of affecting expression or function of the SK2 channel. One exemplary binding assay is described in Example 7. In preferred embodiments, the method further comprises the step of subjecting the test molecule to assays described herein to determine the effect of the test molecule on expression and/or function of the SK2 channel.

The SK2 channel can be provided in any suitable form, as described for other assays herein. In some embodiments, where functional SK2 channel is not required (for example, when binding is itself the characteristic being analyzed), the SK2 channel can comprise SK2 subunit protein, as described elsewhere herein.

According to the invention, binding assays utilize a ligand that is selected to specifically bind the SK2 channel. Any suitable ligand that binds an SK2 channel can be utilized. In some embodiments, the ligand can block SK2 channels; however, such blocking activity is not required in the present invention. Examples of suitable ligands according to the invention include antibodies, peptides, or small molecules. Examples of suitable antibodies include any antibodies that specifically bind the SK2 channel. According to the invention, antibodies can be monoclonal or polyclonal, and can comprise full length proteins or fragments (for example, Fc or F(ab)′).

Many peptides have been identified that bind SK2 channels, and any of these can be utilized in accordance with the present invention. For example, well-investigated specific toxins include, but are not limited to, apamin (isolated from Apis mellifera bee venom); scyllatoxin (isolated from the scorpion Leiurus quinquestriatus); PO5 (isolated from Androctonus maurelanicus); a toxin (isolated from Tityus serrulatus); BmPO5 (isolated from Buthus nmartensii Karsch); PO1; BmPO1; maurotoxin (isolated from Scorpio maurus); Pil (isolated from Pandinus imperator); iberiotoxin (isolated from Mesobuthus taniulus); tamulustoxin (isolated from Mesobuthus tamulus); tamapin (isolated from Mesobuthus tamulus); and the like. The plant alkaloid d-tubocurarium (dTC) can also be used according to the invention.

Examples of small molecules include 1-ethyl-2-benzimidazolinone (EBIO); chlorzoxazone; bisquinolinium cyclophane; dequalinium; low potency antagonists including carbamazepine, chlorpromazine, cyproheptadine, imipramine, and trifluperazine; curare; quaternary salts of bicuculline; and the like.

The affinity of the ligand for the SK2 channel can affect the sensitivity of the assay. For example, a high affinity ligand may not allow the detection of weakly binding test molecules; on the other hand, a low affinity ligand could lead to increased detection of non-specific binding. Thus, in preferred embodiments, the affinity of the ligand is selected to be within a desired range such that the EC₅₀ values obtained from the assays have a reasonable correlation to those obtained from such traditional methods as patch-clamping. For example, the affinity of the ligand for the SK2 channel is preferably selected to be in the range of about 10 pM to about 10 nM, more preferably in the range of about 10 pM to about 1 nM.

According to the invention, the ligand is coupled with a suitable label. A wide variety of labels can be used to label the ligand selected to specifically bind the SK2 channel. Suitable labels can comprise labels that can be visualized via direct detection or indirect detection. Examples of labels that can be visualized via direct detection include, but are not limited to, radioactive isotopes (for example, ¹²⁵I), luminescent materials, materials that utilize optical or electron density, and the like. Examples of labels that can be visualized via indirect detection methods include, but are not limited to, epitope tags (such as FLAG epitope), enzyme tags (such as horseradish peroxidase and alkaline phosphatase), and the like. Labels suitable for use with a corresponding ligand are well known in the art, and the specific type of label utilized according to the invention is not critical.

Preferably, sample is incubated with the labeled ligand under conditions sufficient to allow the labeled ligand to bind the SK2 channel. In some embodiments, the amount of time required for contact with the labeled ligand can be empirically determined by running a time course with a known SK2 modulator, such as apamin, and measuring cellular changes as a function of time.

Separation of unbound labeled ligand from SK2 channel can be accomplished in a variety of ways. In some preferred embodiments, at least one of the components of the assay is immobilized on a solid substrate, from which unbound components can be easily separated (for example, by washing). Suitable solid substrate can be fabricated from a wide variety of materials and in a variety of formats. For example, solid substrates can be utilized in the form of microtiter plates, microbeads (including polymer microbeads, magnetic beads, and the like), dipsticks, resin particles, chromatographic columns, filters, and other like substrates commonly utilized in assay formats. The particular format of the solid substrate is not critical to the invention. The solid substrate is preferably chosen to maximize signal-to-noise ratios, primarily to minimize background binding, as well as for ease of separation of reagents and cost.

Separation of unbound ligand from SK2 channel can be accomplished, for example, by removing a solid substrate (for example, a bead or dipstick) from a reservoir, emptying or diluting a reservoir such as a microtiter plate well, and/or rinsing the solid substrate with a wash solution or solvent. In preferred embodiments, the separation step includes multiple rinses or washes. In embodiments where the solid substrate is a magnetic-bead, the beads can be washed one or more times with a washing solution and isolated using a magnet.

Suitable solution for washing or rinsing typically includes those components of the reaction mixture that do not participate in specific binding such as, for example, salts, buffer, detergent, non-specific protein, and the like.

The label of the labeled ligand can be detected utilizing a variety of techniques, depending upon the nature of the label and other assay components. In some embodiments, the label can be detected while bound to the solid substrate. Alternatively, the label can be detected subsequent to separation from the solid substrate. Detection methodologies are well known in the art and will not be described in further detail.

In some preferred embodiments, ¹²⁵I-apamin binding can be combined with autoradiography of tissue sections (Kuhar M J et al. (1986), Annu Rev Neurosci, 9:27-59).

The various assay systems and methods of the invention can be utilized in conventional laboratory format or adapted for high throughput. Generally, high throughput refers to an assay design that allows easy screening of multiple samples simultaneously and the capacity for robotic manipulation. In preferred embodiments, the inventive assay methods and systems are optimized to reduce reagent usage, or minimize the number of manipulations in order to achieve the analysis desired. Examples of preferred assay formats include 96-well and 384-well plates, levitating droplets, and “lab on a chip” microchannel chips used for liquid handling experiments.

Animal models for neuropathic pain can be used to determine the effect of test molecules on expression of SK2 mRNA, SK2 protein, and/or SK2 channel activity. The screening for neuropathic pain phenotype can include assessment of characteristics including, but not limited to, analysis of molecular markers (for example, expression of SK2 gene products in DRG), assessment of behavioral symptoms associated with neuropathic pain, and detection of cellular degeneration (for example, characterized by Wallerian degeneration and other characteristics described herein).

In further embodiments, methods of the invention can involve measurement of pain responses in mice. For example, in established neuropathic pain models, such as the SNL model, such behaviors as abnormalities (for example, deformity) of the affected limb, posture, gait, and general behavior (for example, aggressive behavior when other rats touch the affected limb on the operated side, increased fighting, sudden licking of the affected limb while at rest, followed by immobility for a few seconds without any apparent external stimuli and the like); foot withdrawal response to repeated mechanical stimuli; and foot withdrawal response to noxious thermal stimuli, can be observed and correlated with SK2 expression, SK2 channel activity, and/or neuropathic pain.

In some embodiments, methods of identifying compounds useful for treating neuropathic pain include assessment of symptoms of neuropathic pain. Assessment of pain can be done in a variety of ways, including behavioral and electrophysiological assessment, the latter providing “surrogate” outcomes. “Surrogate” assessments attempt to correlate physiological findings with behavior. Among the best studied surrogate responses are electrophysiological responses of (1) primary afferent neurons, and (2) spinotbalamic tract neurons in the dorsal horn of the spinal cord.

According to the invention, any of the established neuropathic pain models can be utilized to assay for compounds useful for treating neuropathic pain. Accordingly, preferred embodiments of the invention comprise any of the described methods herein, in combination with the additional step of administering a test molecule identified in one or more of the assays described herein to an animal, such as a neuropathic pain model, and observing affects of the test molecule on the above characteristics.

Although reference is made herein to the SNL animal model for study of neuropathic pain, any suitable accepted model can be utilized in connection with the teachings herein. Animal models for pain include a variety of preclinical animals that exhibit pain syndromes. Commonly studied rodent models of neuropathic pain include the chronic constriction injury (CCI, or Bennett) model; neuroma or axotomy models; the spinal nerve ligation (SNL, or Chung) model; and the partial sciatic transection or Seltzer model (Shir et al., Neurosci. Lett., 115:62-67 (1990)). Exemplary neuropathic pain models include several traumatic nerve injury preparations (Bennett et al., Pain 33: 87-107 (1988); Decosterd et al., Pain 87: 149-58 (2000); Kim et al., Pain 50: 355-363 (1992); Shir et al., Neurosci Lett 115: 62-7 (1990)), neuroinflammation models (Chacur et al., Pain 94: 231-44 (2001); Milligan et al., Brain Res 861: 105-16 (2000)) diabetic neuropathy (Calcutt et al., Br J Pharmacol 122: 1478-82 (1997)), virally induced neuropathy (Fleetwood-Walker et al., J Gen Virol 80: 2433-6 (1999)), vincristine neuropathy (Aley et al., Neuroscience 73: 259-65 (1996); Nozaki-Taguchi et al., Pain 93: 69-76 (2001)), and paclitaxel neuropathy (Cavaletti et al., Exp Neurol 133: 64-72 (1995)).

In another aspect, the invention provides a method of hyperpolarizing a cell comprising contacting a cell with a hyperpolarization effective amount of a composition that increases current mediated by SK2 channels in the cell.

It can be desirable to hyperpolarize a cell under certain conditions. For example, in certain cell lines, where the resting membrane potential is relatively low (approximately −40 mV), hyperpolarization can activate some membrane channels under physiological conditions. Such cell lines include, for example, CHO cells, tsA201 cells, or HEK293 cells. In these embodiments, activation of ion channels expressed within these cells that undergo voltage dependent steady state inactivation can require a more negative membrane potential to shift them into closed conformation states from which they can be activated. Hyperpolarization by activation of SK2 channels can then allow subsequent activation of depolarization activated channels that undergo steady state inactivation such as fast inactivating sodium channels and T-type calcium channels. Therefore, hyperpolarization of these types of cells can activate some channels under physiological conditions. Hyperpolarization by activation of SK2 channels can also activate channels that are activated by hyperpolarization such as hyperpolarization-activated non-selective cation channels.

Compositions useful for hyperpolarizing a cell comprise compounds that activate or enhance expression of SK2 protein, or increase ion flux through an SK2 channel. Such compounds, and methods of identifying such compounds, are described herein.

The hyperpolarization effective amount is the amount of an active composition that is effective to hyperpolarize the cell when administered to the cell. Generally, a hyperpolarization effective amount of an active composition will cause the membrane potential to become more negative. The hyperpolarization effective amount of an active composition can be readily determined by those skilled in the art by measuring the membrane potential of the cell, using methods well known in the art (for example, patch-clamp techniques, voltage sensitive dyes, and the like). Hyperpolarization toward the equilibrium potential for K⁺ ions is typically approximately −90 mV under physiological conditions.

Methods of hyperpolarizing a cell described herein can be applied to any type of cells. Examples of suitable cells include cells that do not natively express SK2 channel (including, but not limited to, tsA201, HEK293, CHO, and lymphocytes), and native SK2 expressing cells (including, but not limited to, cells described herein as naturally-occurring cells that express SK2, and non-excitable cells such as lymphocytes.

In one aspect, the invention provides methods for preventing the onset of neuropathic pain in a subject, comprising administering to the subject a composition that increases ion flow through SK2 channels, the composition administered to the subject in a prophylactically effective amount. In preferred embodiments, the method is useful when applied prior to a painful event, for example, prior to chemotherapy or a surgery that is known or suspected to result in neuropathic pain.

As used herein, the term “composition” is intended to encompass a product comprising the specified compounds in the specified amounts, as well as any product that results, directly or indirectly, from combinations of the specified compounds in the specified amounts.

As used herein, a “prophylactically effective amount” refers to that amount of active compound that inhibits the onset of neuropathic pain in a subject. Methods are known in the art for determining the prophylactically effective amount of an active compound.

The invention further provides methods for treatment of neuropathic pain in a subject in need thereof comprising administering a composition that increases ion flow through SK2 channels, the composition administered to the subject in a therapeutically effective amount. Preferably, the composition is administered to a sensory neuron.

In some preferred embodiments, the invention provides methods for treatment of neuropathic pain in a subject in need thereof comprising administering a composition that increases ion flow through SK2 channels, the composition administered to the subject in an SK2 channel-opening amount.

Examples of suitable compounds that could be included in the composition for treatment of neuropathic pain include 1-ethyl-2-benzimidazolinone (1-EBIO), and 2-amino-5-chlorobenzoxazole (zoxazolamine). 1-EBIO has been shown to enhance activity of intermediate conductance Ca²⁺-activated K⁺ channels. Zoxazolamine is structurally similar to 1-EBIO. These two compounds have recently been demonstrated to enhance SK2 channel activity. The order of potency of these compounds is 1-EBIO>zoxazolamine. See Cao, Y-J. et al., JPET 296:683-689 (2001). These compounds activate SK2 channels in the nominal absence of intracellular Ca²⁺ in whole-cell experiments, and these compounds allow for channel activation at [Ca²⁺] of as low as 20 nM.

As used herein, a “therapeutically effective amount” refers to that amount of an active composition alone, or together with other analgesics, that produces the desired reduction of pain in a subject. In the case of treating a condition characterized by decreased SK protein expression, the desired reduction of pain is associated with increased SK protein expression and/or ion flux through an SK2 channel to a level that is within a normal range found in a control individual not suffering from neuropathic pain. During treatment, such amounts will depend upon such factors as the particular condition being treated, the severity of the condition, the individual patient parameters including age, physical condition, size and weight, the duration of the treatment, the nature of the particular agent thereof employed and the concurrent therapy (if any), the specific route of administration and like factors within the knowledge and expertise of the health practitioner. A physician or veterinarian of ordinary skill can readily determine and prescribe the effective amount of the compound required to treat and/or prevent the progress of the condition.

In another aspect, the invention provides a method for treating neuropathic pain in a subject in need thereof, comprising administering to the subject a composition that increases the open probability of SK2 channels in a sensory neuron of the subject, the composition administered to the subject in a therapeutically effective amount. The open probability of an SK2 channel refers to the fraction of time the SK2 channel stays in the open conformation, thus allowing passage of ions across the membrane. Suitable compositions for increasing the open probability of SK2 channels can open the channel pore, destabilize non-conducting states of the channel, and/or shift the Ca²⁺ dependence of activation in the sensory cells of the subject. Test molecules for use in such compositions can be identified utilizing the methods described herein.

In another aspect, the invention provides methods for treating neuropathic pain in a subject in need thereof, comprising administering to the subject a composition that increases the number of functional SK2 channels in sensory cells of a subject, the composition administered to the subject in a therapeutically effective amount. Preferably, the method involves a composition that increases the expression of SK proteins in sensory cells of the subject, most preferably neurons. Examples of suitable compositions include compounds that increase and/or enhance SK transcription and/or translation, and/or decrease or inhibit degradation of SK2 expression products, which can be identified by methods described herein. In other embodiments, nucleic acid molecules encoding functional SK proteins, or an active fragment of an SK protein, can be used to increase expression of SK protein as described herein.

DNA molecules capable of encoding active SK proteins can be administered to the subject via transplanting into the subject a cell (for example, a sensory neuron) genetically modified to express a SK protein or an active fragment thereof. Transplantation can provide a continuous source of sufficient SK channel, thus, sustained alleviation of neuropathic pain. For a subject suffering from prolonged or chronic neuropathic pain, such a method can, in some embodiments, have the advantage of obviating or reducing the need for repeated administration of analgesics. Such a method can be useful to alleviate neuropathic pain as described for the transplantation of cells that secrete substances with analgesic properties (see, for example, Czech and Sagen, Prog. Neurobiol. 46:507-529 (1995)).

Using methods well known in the art, a sensory neuron cell readily can be transfected with an expression vector containing a nucleic acid encoding an SK protein (see, for example, Chang, (1995), Somatic Gene Therapy, CRC Press, Boca Raton). Preferably, the neuron cell is immunologically compatible with the subject. For example, a particularly useful cell is a cell isolated from the subject to be treated, since such a cell is immunologically compatible with the subject. A cell derived from a source other than the subject to be treated also can be useful if protected from immune rejection using, for example, such techniques as microencapsulation or immunosuppression. Useful microencapsulation membrane materials include alginate-poly-L-lysine alginate and agarose (see, for example, Tai and Sun, FASEB J. 7:1061 (1993)). For example, pain reduction has been achieved using polymer encapsulated cells transplanted into the rat spinal subarachnoid space (Wang et al., Soc. Neurosci. Abstr. 17:235 (1991)). For treatment of a human subject, the cell can be a human cell, although a non-human mammalian cell also can be useful. Considerations for neural transplantation are described (for example, in Chang, supra, 1995).

A cell derived from the nervous system can be particularly useful for transplantation to the nervous system since the survival of such a cell is enhanced within its natural environment. A neuronal precursor cell is particularly useful in the method of the invention since a neuronal precursor cell can be grown in culture, transfected with an expression vector and introduced into an individual, where it is integrated. The isolation of neuronal precursor cells, which are capable of proliferating and differentiating into neurons and glial cells, is described in Renfranz et al., Cell 66:713-729 (1991).

Methods of transfecting cells ex vivo are well known in the art (Kriegler, Gene Transfer and Expression: A Laboratory Manual, W. H. Freeman & Co., New York (1990)). For the transfection of a cell that continues to divide such as a neuronal precursor cell, a retroviral vector is preferred. For the transfection of an expression vector into a postmitotic cell such as a neuron, a replication-defective herpes simplex virus type 1 (HSV-1) vector is useful (Palmer J A et al., J Virology 74:5604-5618 (2000)).

A nucleic acid encoding a full length of a SK protein or an active fragment thereof can be expressed under the control of one of a variety of promoters well known in the art, including a constitutive promoter or inducible promoter (see, for example, Chang, supra, 1995). Particularly useful constitutive promoters for high-level expression include the Moloney murine leukemia virus long-terminal repeat (MLV-LTR), the cytomegalovirus immediate-early (CMV-IE), and the simian virus 40 early region (SV40). Nucleic acid sequences encoding active SK2 proteins are known, as discussed herein. Other examples of nucleic acid sequences encoding an active SK2 protein is disclosed herein, such as SEQ ID NO:1 and SEQ ID NO:2.

Numerous transfection and transduction techniques as well as appropriate expression vectors are well known to those of ordinary skill in the art. In vivo gene therapy uses vectors such as adenovirus, retroviruses, vaccinia virus, bovine papilloma virus, and herpes virus such as Epstein-Barr virus. Gene transfer can also be achieved using non-viral means requiring infection in vitro. According to this particular embodiment, calcium phosphate, DEAE dextran, electroporation, and protoplast fusion can be included. Targeted liposomes may also be potentially beneficial for delivery of DNA into a cell.

DNA molecules capable of encoding active SK proteins can also be administered to the subject via direct injection or surgical implantation in the proximity of the damaged tissues or cells in order to avoid the problems presented by brain/blood barrier. Successful delivery to the central nervous system by direct injection or implantation has been documented. See, for example, Otto et al., J. Neurosci. Res., 22: 83-91 (1989); Goodman & Gilman's The Pharniacological Basis of Therapeutics, 6th ed, pp 244; Williams et al., Proc. Natl. Acad. Sci. USA 83: 9231-9235 (1986); and Oritz et al., Soc. Neurosci. Abs. 386: 18 (1990).

In another aspect, the invention provides a method of identifying a molecule useful for treating neuropathic pain comprising steps of: (a) providing cells that express SK2; (b) contacting the cells with a membrane potential sensitive fluorescent dye; (c) contacting the cells with a test molecule; (d) obtaining information indicative of a change in membrane potential in response to the test molecule; (e) contacting the cells with a specific inhibitor of the SK2 channel; and (f) determining whether the change in membrane potential is blocked by the specific inhibitor. Exemplary specific inhibitors of SK2 channels are described elsewhere herein.

Preferably, the invention provides a method of identifying a compound useful for treating neuropathic pain comprising steps of: (a) providing cells capable of expressing SK2; (b) contacting the cells with a membrane potential sensitive fluorescent dye; (c) contacting the cells with a test molecule; (c) contacting the cells with CsCl; and (d) obtaining information indicative of a change in membrane potential of the cells toward equilibrium potential of Cs⁺ (Ecs) that is elicited by the test molecule compared to a control. An increased depolarization of the cells compared to the control indicates that the test molecule is an activator or enhancer for the SK2 channel, and a decreased depolarization compared to control indicates that the test molecule is an inhibitor for the SK2 channel. Preferably, the CsCl is provided to the cells in a Cs⁺ containing buffer.

In some preferred embodiments, the assay methods and systems described herein further comprise contacting the cells with an SK2 channel activator prior to contacting the cells with CsCl. According to these particular embodiments, addition of the SK2 channel activator results in hyperpolarization of the cell membrane. The SK2 channel activator may, but does not necessarily, have the effect of increasing intracellular Ca²⁺ levels that normally open SK2 channels. Therefore, an SK2 channel activator can cause membrane hyperpolarization independent of intracellular Ca²⁺ levels, or membrane hyperpolarization dependent upon increased Ca²⁺. In both cases, an increased Cs⁺-induced depolarization of the membrane compared to vehicle control will be observed. An inhibitor of the SK2 channel, however, will cause a decreased Ca²⁺ evoked hyperpolarization after exposure to agents that increase intracellular Ca²⁺ levels, and a decreased Cs⁺-induced depolarization compared to control.

In some preferred embodiments, the assay methods and systems described herein further comprise contacting the cells with compounds that increase intracellular Ca²⁺ levels of the cells. Suitable methods to increase intracellular Ca²⁺ levels include, but are not limited to, extracellular addition of ATP and thapsigargin; activation of other GPCR receptors coupling to Gq and other G proteins activating PLC and ultimately causing the release of Ca²⁺ from intracellular stores; release of intracellular Ca²⁺ by activators of Ca²⁺ channels located on membranes of intracellular stores; depletion of intracellular stores by other blockers of Ca²⁺ re-uptake into intracellular stores; activation of plasma membrane calcium channels by organic openers (for example, BAYK8644); and addition of Ca²⁺ ionophores such as ionomycin and A23187.

In yet another aspect, the invention provides combination therapy for preventing the onset of or treating neuropathic pain comprising any of the treatment methods described herein, in combination with administering one or more additives, such as analgesics or adjuvants. Suitable additives include, but are not limited to, morphine or other opiate receptor agonists; nalbuphine or other mixed opioid agonist/antagonists; tramadol; baclofen; clonidine or other alpha-2 adrenoreceptor agonists; amitriptyline or other tricyclic antidepressants; gabapentin or pregabalin, carbamazepine, phenytoin, lamotrigine, or other anticonvulsants; and/or lidocaine, tocainide, or other local anesthetics/antiarrhythmics. For example, known analgesics, such as chlorzoxazone, can be utilized in combination with molecules identified according to the invention.

In another aspect, the present invention provides methods of creating an animal model of neuropathic pain. In one embodiment, the method comprises administering to an animal, preferably a rodent, a neuropathic pain effective amount of a composition that reduces the current mediated by SK2 channels in a sensory neuron in the animal.

According to this particular embodiment, the method of creating an animal model of neuropathic pain preferably involves a composition that decreases the expression of SK2 proteins. Examples of such compositions include compounds that decrease SK2 transcription or translation, which can be identified by methods described herein. In some embodiments, antisense nucleic acids or small interference RNA (siRNA) can also be used to reduce the expression of SK2 proteins.

Antisense based strategies can be used to create the animal pain model by reducing expression of SK2 in sensory neuron cells. The principle is based on the hypothesis that sequence-specific suppression of gene expression can be achieved by intracellular hybridization between mRNA and a complementary antisense species. The formation of a hybrid RNA duplex can then interfere with the processing/transport/translation and/or stability of the target SK2 mRNA. Hybridization is required for the antisense effect to occur. Antisense strategies can use a variety of approaches, including the use of antisense oligonucleotides, injection of antisense RNA, and transfection of antisense RNA expression vectors. Phenotypic effects induced by antisense effects are based on changes in criteria such as protein levels, protein activity measurement, and target mRNA levels.

An antisense nucleic acid can be complementary to an entire coding strand of a SK2 gene, or to only a portion thereof. An antisense nucleic acid molecule can also be complementary to all or part of a non-coding region of the coding strand of an SK2 gene. The non-coding regions include the 5′ and 3′ sequences that flank the coding region (“5′ and 3′ untranslated regions”) and introns, and are not translated into amino acids. Preferably, the non-coding region is a regulatory region for the transcription or translation of the SK2 channel gene.

An antisense oligonucleotide of the invention is complementary to the nucleotide sequence of SK2 and can be, for example, about 15, 20, 25, 30, 35, 40, 45 or 50 nucleotides or more in length. Preferably, the antisense oligonucleotide is complementary to the nucleotide sequence of hSK2, more preferably SEQ ID NO: 1 or SEQ ID NO: 2. An antisense nucleic acid can be constructed using chemical synthesis and enzymatic ligation reactions using procedures known in the art. For example, an antisense nucleic acid (for example, an antisense oligonucleotide) can be chemically synthesized using naturally occurring nucleotides or modified nucleotides designed to increase the biological stability of the molecules or to increase the physical stability of the duplex formed between the antisense and sense nucleic acids. In one embodiment, phosphorothioate derivatives and acridine substituted nucleotides can be used. Examples of modified nucleotides that can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxytnethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methyleytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2- methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

In some embodiments, an antisense nucleic acid molecule can be a CC-anomeric nucleic acid molecule. A CC-anomeric nucleic acid molecule forms specific double-stranded hybrids with complementary RNA in which the strands run parallel to each other (Gaultier et al. Nucleic Acids Res. 15:6625-664 1 (1987)). The antisense nucleic acid molecule can also comprise a 2′-o-methylribonucleotide (Inoue et al. Nucleic Acids Res. 15:6131-6148 (1987)) or a chimeric RNA-DNA analogue (Inoue et al. FEBS Lett. 215:327-330 (1987)).

Alternatively, the antisense nucleic acid can be produced biologically using an expression vector into which a nucleic acid has been subcloned in an antisense orientation (for example, RNA transcribed from the inserted nucleic acid will be of an antisense orientation to a target nucleic acid of interest). According to this embodiment, a DNA molecule is operably linked to a regulatory sequence in a manner that allows for expression (by transcription of the DNA molecule) of an RNA molecule that is antisense to the mRNA encoding a SK protein. Regulatory sequences operably linked to a nucleic acid cloned in the antisense orientation can be chosen which direct the continuous expression of the antisense RNA molecule in a variety of cell types, for instance viral promoters and/or enhancers. Alternatively, regulatory sequences can be chosen that direct constitutive, tissue specific or cell type specific expression of antisense RNA.

According to the invention, the antisense expression vector can be in the form of a recombinant plasmid, phagemid or attenuated virus in which antisense nucleic acids are produced under the control of a high efficiency regulatory region, the activity of which can be determined by the cell type into which the vector is introduced. Suitable viral vectors include retrovirus, adenovirus, adeno-associated virus, herpes virus, vaccinia virus, polio virus and the like. For a discussion of the regulation of gene expression using antisense genes see Weintraub et al., Trends in Genetics, 1:22-25 (1985).

The antisense nucleic acid molecules of the invention are typically administered to a subject or generated in situ such that they hybridize with or bind to cellular mRNA and/or genomic DNA encoding an SK2 protein to thereby inhibit expression of the protein, for example, by inhibiting transcription and/or translation. The hybridization can be by conventional nucleotide complementarity to form a stable duplex. Alternatively, in the case of an antisense nucleic acid molecule that binds to DNA duplexes, hybridization can be through specific interactions in the major groove of the double helix. An exemplary route of administration of antisense nucleic acid molecules of the invention includes direct injection at a tissue site. Alternatively, antisense nucleic acid molecules can be modified to target selected cells and subsequently administered systemically. For example, for systemic administration, antisense molecules can be modified such that they specifically bind to receptors or antigens expressed on a selected cell surface, for example, by linking the antisense nucleic acid molecules to peptides or antibodies that bind to cell surface receptors or antigens. The antisense nucleic acid molecules can also be generated in situ by expression from vectors described herein harboring the antisense sequence. To achieve sufficient intracellular concentrations of the antisense molecules, vector constructs in which the antisense nucleic acid molecule is placed under the control of a strong polymerase II or polymerase III promoter are preferred.

In a preferred embodiment, the method of creating a neuropathic pain model involves small interfering RNA (siRNA). According to this particular embodiment, introduction of double-stranded RNA is utilized to suppress gene expression through a process known as RNA interference. Many organisms possess mechanisms to silence any gene when double-stranded RNA (dsRNA) corresponding to the gene is present in the cell. The technique of using dsRNA to reduce the activity of a specific gene was first developed using C. elegans and has been termed RNA interference, or RNAi (Fire, et al., Nature 391: 806-811 (1998)). RNAi has since been found to be useful in many organisms, and recently has been extended to mammalian cells in culture (see review by Moss, Curr Biol., 11 (19):R772-5 (2001), and references therein).

RNAi has been shown to involve the generation of small RNAs of 21-25 nucleotides (Zamore, et al., Cell 101:25-33 (2000); and Hammond, et al, Nature 404: 293-296 (2000)). These small interfering RNAs, or siRNAs, are initially derived from a larger dsRNA that begins the process, and are complementary to the target RNA that is eventually degraded. The siRNAs are themselves double-stranded with short overhangs at each end; they act as guide RNAs, directing a single cleavage of the target in the region of complementarity (Zamore supra; Elbashir et al., Genes Dev 15: 188-200 (2001)).

Some exemplary methods of producing siRNA, 21-23 nucleotides in length from an in vitro system, as well as methods of utilizing the siRNA to interfere with mRNA of a gene in a cell or organism are described in WO0175164 A2.

In some embodiments, the siRNA can be made in vivo from a mammalian cell using a stable expression system. The pSUPER vector system, which directs the synthesis of small interfering RNAs (siRNAs) in mammalian cells can be utilized for this purpose (Thijn et al., Science, 296: 550-553 (2002)). Briefly, the pSUPER vector system is constructed by cloning the H1-RNA promoter in front of the gene specific targeting sequence (19-nt sequences from the target transcript separated by a short spacer from the reverse complement of the same sequence). Five thymidines (T5) are also cloned into the vector as termination signal. The resulting transcript is predicted to fold back on itself to form a 19-base pair stem-loop structure, resembling that of C. elegans Let-7. The size of the loop (the short spacer) is preferably 9 bp. A small RNA transcript lacking a poly-adenosine tail containing a well-defined start of transcription and a termination signal consisting of five thymidines in a row (T5) was produced from the vector. Most importantly, the cleavage of the transcript at the termination site occurs after the second uridine, yielding a transcript resembling the ends of synthetic siRNAs, which also contain two 3′ overhanging T or U nucleotides (nt). The siRNA expressed from pSUPER is capable of down-regulating gene expression as efficiently as the synthetic siRNA.

Thus, in one embodiment, the invention provides a method of creating a neuropathic pain model, comprising steps of: (a) providing siRNA which targets the mRNA of the SK2 gene for degradation to a cell or organism; and (b) maintaining the cell or organism produced in (a) under conditions under which siRNA interference of the mRNA of the SK2 gene in the cell or organism occurs. The siRNA can be produced chemically via nucleotide synthesis, from an in vitro system similar to that described in WO0175164, or from an in vivo stable expression vector similar to pSUPER described herein. The siRNA can be administered similarly as that of the anti-sense nucleic acids described herein.

In another aspect, the invention provides a method of creating an animal model of neuropathic pain comprising administering a composition that decreases ion flux through the SK2 channel in sensory neuron cells. Examples of compounds that can be included in the composition include apamin, bisquinolinium cyclophane UCL-1684 (Stroebaek, et al., Br. J. Pharmacol. 129: 991-999, (2000); and Fanger et al., J. Biol. Chem. 276:12249-12256, (2001)), or peptide toxin Leiurotoxin I (Lei, also known as scyllatoxin) (Hanselmann et al., J. Physiol. 496:627-637, (1996); and Stroebaek, et al., supra). Preferably, the composition is a Lei analog, Lei-Dab⁷, which specifically blocks SK2 channel with a K_{d} of 3.8 nM (Shakkottai et al, J. Biol. Chem., 276: 43145-43151, (2001)). In some embodiments, other compounds that decrease ion flux through the SK2 channels in DRG cells can be administered, and these compounds can be identified by methods described supra.

The following examples illustrate the present invention without, however, limiting the same thereto.

### EXAMPLES

### Reagents and Methods

The following reagents are used in the Examples:

| | |
|---|---|
| Buffer 1 | 100 mM Tris, 150 mM NaCl, pH 7.5 |
| Buffer 3 | 100 mM Tris, 10 mM NaCl, 50 mM MgCl, pH 9.5 |

### Example 1

### Expression of SK2 in Neuropathic Pain Model

This example illustrates decreased expression levels of SK2 mRNA and protein in neurons isolated from dorsal root ganglia of a neuropathic pain model.

### Preparation of Animal Model

Male Harlan Sprague-Dawley rats weighing 100-150 grams were housed in cages with solid bottoms and sawdust bedding, with a 12 hour/12 hour reversed light cycle (lights on 2100-900), and allowed free access to food pellets and water. The rats were kept at least 7 days under these conditions prior to surgery. Animals were housed in groups of two after surgical interventions.

A surgical neuropathy was performed using procedures described in Pain 50(3): 355-363 (Kim and Chung, 1992). The resulting animal model is commonly referred to as the SNL model, or spinal nerve ligation model, or the Chung model. Under isoflurane/oxygen anesthesia, the rat was placed in a prone position, and a dorsal midline incision was made from approximately L3-S2 levels. Using a mixture of sharp and blunt dissection, the left L6/S1 posterior interarticular process was exposed and resected to permit adequate visualization of the L6 transverse process, which was gently removed. Careful teasing of the underlying fascia exposed the left L4 and L5 spinal nerves distal to their emergence from the intervertebral foramina. The nerves were gently separated, and the L5 and L6 nerves were firmly ligated with 6-0 silk suture material. For in vivo/ex vivo electrophysiological studies, the L4 and L5 nerves were ligated with 6-0 silk suture material. The wound was then inspected for hemostasis and closed in two layers. No surgical procedure was done on the right side.

For the control group (sham operation for spinal nerve ligations), the surgical procedure was identical to that of the experimental group, except that spinal nerves were not ligated. A sham operation was performed on the left side, and no surgical procedure was done on the right side.

After surgery, the rats were returned to their pre-operative location and maintained under the same conditions as during the pre-operative period. Calibrated Von Frey filaments (0.4-15.1 g) were used to document paw thresholds at 4 days, 1, 2, 4 and 5 weeks post-surgery. Immediately after behavioral testing, dorsal root ganglia (DRG) were harvested. Rats were anesthetized with isoflurane/oxygen, underwent a quick trans-cardiovascular phosphate buffer saline perfusion, right and left side L5/6 DRG were removed and flash frozen on dry ice for RNA extraction (4 days and 5 week post-surgery rats), or in OCT embedding medium (VWR) for sectioning (1, 2 and 4 week post-surgery rats, left and right L5 DRG were embedded in the same cryomold side by side for comparison). The following tests were performed on the DRG samples.

### RNA Extraction and Amplification

Total RNA was extracted from left L5/L6 for each rat (RNEasy, Qiagen). Conventional first strand cDNA synthesis was performed on 1/10^{th} of the yield using Superscript II (Life Technologies), as per the manufacturer's protocol. These cDNAs were diluted 4-fold in molecular biology grade water containing a final concentration of 10 ng/μl of polyinosine carrier.

### Quantitative PCR

cDNA Samples from SNL and control rats were simultaneously analyzed using an iCycler® (BioRad, Inc.), with Qiagen Taq Master Mix with 1:1000 Sybr Green (Molecular Probes, Inc.) per reaction, according to manufacturer's instructions. For standard curve preparation, conventional end-product PCR products were T/A cloned into pCR® 4-TOPO vector (Invitrogen) using the TOPO TA Cloning® kit (Invitrogen) according to the manufacturer's instructions. Plasmids were then sequenced, quantified by spectrophotometry, and used as a standard dilution series at calculated copy number.

For quantitative PCR of SK2, the following primers were used:

| | |
|---|---|
| 5′-TGGACTGTCC GAGCTTGTGA AAGG-3′ | (SEQ ID NO: 7) |
| 5′-CCTTGGTGGT AGCCGTAGTG GCA-3′ | (SEQ ID NO: 8) |

These primers correspond to bases 982-1005 and 1163-1185 of GenBank sequence #U69882, respectively. The primers were designed to be unique to SK2 as verified by BLAST search, and to include a splice junction for a large intron as deduced by alignment with the human genome draft sequence so as to prevent amplification of contaminating genomic DNA.

Rat cyclophilin A (GenBank access No: NM_{—}017101) was used as a housekeeping gene for normalization purposes. The following primers were used:

| | |
|---|---|
| 5′-TGAGCACTGG GGAGAAAGGA TTTGG-3′ | (SEQ ID NO: 9) |
| 5′-TCGGAGATGG TGATCTTCTT GCTGG-3′ | (SEQ ID NO: 10) |

While these primers did not span an intron-containing region, the abundance of cyclophilin mRNA was sufficient such that genomic DNA contamination introduced only trivial variation for this PCR product.

Two microliters of 4× diluted cDNAs as above were aliquotted in duplicate onto 96-well plates, and assayed separately and simultaneously for SK2 and cyclophilin A. The PCR reaction protocol used was: 10 minutes denaturation at 95° C., followed by 40 cycles of 95° C. for 1 minute, 65° C. for 30 seconds, 72° C. for 30 seconds. This protocol was followed by a melt curve to verify specific melting temperatures of the PCR products.

PCR yielded a 181-bp amplicon of SK2 and a 363 bp DNA fragment of Rat Cyclophilin A. Relative abundance was estimated as fluorescence by gene-specific standard curves using serial dilutions of brain cDNA as template. To compare relative fluorescence, sample loading was corrected using cyclophilin abundance. Cyclophilin fluorescence was ranged to a fraction of 1 by dividing all sample values by the largest sample value, under the assumption that the maximum value was the closest to 100% mRNA retrieval from one DRG and that other sample values were fractional retrieval. Values for SK2 fluorescence were normalized to cyclophilin levels. Normalized values for nerve-ligated DRG and their respective controls were compared using paired T-test (for ipsilateral and contralateral samples from the same rat) or unpaired T-test (for sham operated control samples). P-values <0.05 were considered significant.

### In Situ Hybridization

Frozen sections (10 μm) of L5 DRG (tissue block prepared as above) on Superfrost Plus slides (VWR) were post-fixed in 1× phosphate buffered saline (1×PBS, pH 7.4) with 4% paraformaldehyde for 15 minutes and then rinsed in 1×PBS three times for 10 minutes each rinse. Following 15 minutes equilibration in 5× saline sodium citrate buffer (5×SSC, pH 7), the sections were pre-hybridized for 2 hours at 58° C. in hybridization buffer (50% formamide, 5×SSC, 100 μg/ml salmon sperm DNA).

Sections were then incubated in hybridization buffer with 1 μg/ml digoxigenin (dig)-labeled antisense cRNA probes of rat SK2, overnight at 58° C. The cRNA probe was as follows:

5′-AGCCCCCAGCGTCGGTTGTAGGAGGAGGTGGTGGTGCGTCCTCCCCG
TCTGCTGCCGCCGCCGCCTCATCCTCAGCCCCAGAGATCGTGGTGTCTAA
GCCGGAGCA-3′
(SEQ ID NO: 11, GB # U69882, recognizes sequence
bases 104-208 of rSK2).

Dig-labeled sense probes at the same concentration were used as probe to control for specificity.

Post-hybridization washes were carried out for 15 minutes in 2×SSC (pH 7) at room temperature, 1 hour in 2×SSC at 65° C., 1 hour in 0.1×SSC (pH 7) at 65° C. Following 5 minutes equilibration in Buffer 1, the sections were incubated for 2 hours in Buffer 1 with 1% Boehringer Blocking Reagent and 1:500 diluted AP-coupled anti-Dig antibody (Roche) at room temperature. The sections were then rinsed 3 times with Buffer 1. After rinse, the sections were equilibrated in Buffer 3 for 5 minutes and stained in Buffer 3 with nitroblue tetrazolium chloride and 5-bromo-4-chloro-3-indolyl-phosphate (NBT/BCIP, Roche, Catalog No. 1681451, 4.5 μl of NBT and 3.5 μl of BCIP in 1 ml Buffer 3) over night at room temperature. The sections were rinsed with TE Buffer (10 mM Tris, 1 mM EDTA, pH 8.0) for 10 minutes and followed 60 minutes in 95 % alcohol. After rinse in H₂O, the sections were dehydrated and mounted for microscope examination.

### Immunocytochemistry

L5 DRG (tissue block prepared as above) were sectioned at 10 μm and mounted on Superfrost Plus slides (VWR). Sections were fixed in 1× phosphate buffered saline (1×PBS, pH 7.4) with 4% paraformaldehyde for 10 minutes and then rinsed in 1×PBS three times for 10 minutes each rinse. After incubation in 1×PBS containing 0.3% H₂O₂ for 15 minutes at room temperature, the sections were blocked in 1×PBS containing 0.3% Triton-100 and 5% normal goat for 1 hour at room temperature. Rabbit anti-SK2 antibody (Alomone labs, Cat # APC-028, 1:500 dilution) in 1×PBS containing 0.3% Triton-100 and 5% normal goat serum were applied to section and incubated overnight at 4° C. After 1×PBS rinse for 3 times at 10 minutes each, the sections were incubated with biotinylated goat anti rabbit IgG (Chemicon, Cat # BP132B) at 1:1000 in 1×PBS containing 0.3% Triton-100 and 5% normal goat serum for 1 hour at room temperature. After rinse in 1×PBS the sections were developed with Vectastain Elite ABC kit and DAB (3,3′-diaminobenzidine-tetrhydrochloride) kit (Vector Laboratories) per manufacturer's instructions.

### Results

Quantitative PCR analysis revealed SK2 mRNA expression level was decreased more than 5-fold in DRG neurons of the SNL rat at either 4 days or 5 weeks after surgery. Results are illustrated in FIG. 1. In FIG. 1, SNL rats are represented at bar A, and control rats are represented at bar B. Relative units of mRNA are represented on the Y-axis. Data is shown at 4 days (group I) and 5 weeks (group II). Data represented in FIG. 1 represent the mean values obtained from seven rats.

The results illustrated in FIG. 1 were confirmed by in situ hybridization with an SK2 specific probe. SK2 was expressed in all sized DRG neurons and this expression decreased in ipsilateral (ligated side) DRG neurons compared to the contralateral (non ligated control) DRG neurons of SNL rats. The decreased expression level was observed for 4 weeks post-surgery. In addition, immunohistochemical analysis revealed that SK2 protein levels were similarly decreased in ipsilateral (ligated side) DRG neurons compared to the contralateral (non ligated control) DRG neurons of SNL rats.

### Example 2

### Ex Vivo Recording from DRG Neurons

This example illustrates an ex vivo methodology for measurement of spontaneous discharges from DRG neurons.

For in-vitro studies, the left L4 and L5 spinal nerves are ligated as described in Example 1. Seven to 21 days later, animals are anesthetized with isoflurane (3% in oxygen (O₂)) and the L4 and L5 dorsal root ganglia (DRGs), along with dorsal roots and spinal nerves, are removed. The DRG are placed in an in-vitro recording chamber that consists of two separate compartments: one for the dorsal root and the other for the DRG and spinal nerve. The compartment containing DRG and spinal nerve is perfused with oxygenated (95% O₂ and 5% CO₂) artificial cerebrospinal fluid (ACSF) (composition in mM: NaCl 130, KCl 3.5, NaH₂PO₄ 1.25, NaHCO₃ 24, Dextrose 10, MgCl₂ 1.2, CaCl₂ 1.2, pH 7.3) at a rate of 4-5 ml/minute. The dorsal root compartment is filled with mineral oil. The temperature is maintained at 35° C. (±1° C.) through a temperature controlled water bath.

The spinal nerve is stimulated using a suction electrode, and spontaneous discharges are recorded from the teased dorsal root fascicles. Fiber types are classified according to their conduction velocity: >14 m/sec for Aβ, 2-14 m/sec for Aδ, and <2 m/sec for C fibers (Harper et al., 1985; Ritter et al., 1992; Waddel et al., 1990). For analysis of the effects of various compounds, the number of spikes per minute is calculated and the numbers are compared before and after a perfusion of a compound.

### Example 3

### Cloning of Human SK2 A⁺ and SK2 A Isoforms

Human SK2 cDNA sequence was identified from NCBI Genbank human genome draft sequence using rat SK2 cDNA (GenBank Access No. U69882) coding region as the query. The human SK2 gene was found in a human genomic contig (Genbank Accession No. NT_{—}034772.4) located in chromosome 5. The complete coding region of human SK2 cDNA was then amplified by PCR reaction from combined human spinal cord and dorsal root ganglion (DRG) cDNA libraries using two primers:

forward primer,

(SEQ ID NO: 5)

5′ AC GAT GAA TTC GCC ACC ATG AGC AGC TGC AGG TAC
AAC G 3′,
and
reverse primer,

(SEQ ID NO: 6)

5′ ACG ACT ACT CGA GCT AGC TAC TCT CTG ATG AAG TTG
GT 3′.

The PCR reaction was performed at 94° C. 40 seconds, 65° C. 40 seconds, 72° C. 3 minutes, for 35 cycles.

The resulting DNA was then cloned into the mammalian expression vector pcDNA 3.1/Zeo between EcoR1 and Xho1 sites. The completed insert region was then sequenced using an automated DNA sequencer (PE Prizm 337 DNA sequencer).

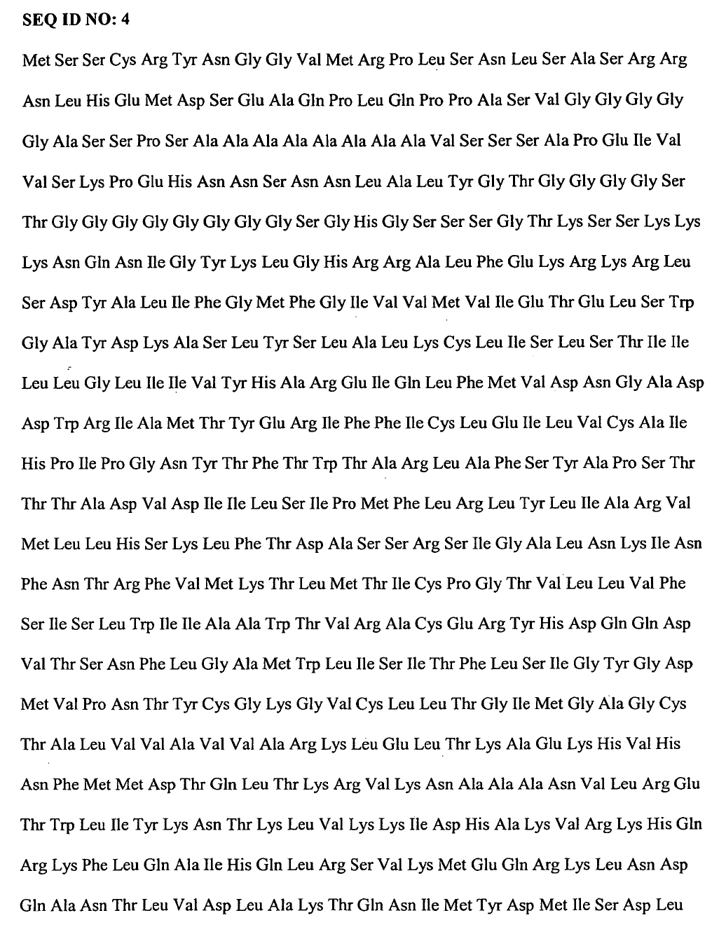

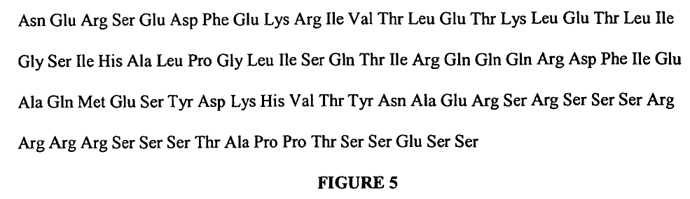

Two clones were identified, SEQ ID NO: 1 (FIG. 2) and SEQ ID NO: 2 (FIG. 4), and they encode for two proteins SEQ ID NO: 3 (FIG. 3) and SEQ ID NO: 4 (FIG. 5), respectively. The two nucleic acid sequences are identical except for an in-frame insertion of 3 nucleotides at nucleotide position 173, coding for an alanine at amino acid position 58. The clone found to contain a single codon (alanine) insertion was thus referred to as hSK2 A⁺ (SEQ ID NO: 1), and the other was referred to as hSK2 A⁻ (SEQ ID NO: 2). hSK2A⁺ was 1743 nucleotides in length and encoded a polypeptide of 580 amino acids. hSK2A⁻ was 1740 nucleotides in length and encoded a polypeptide of 579 amino acids.

ClustalW Multiple Alignment revealed that the coding sequences of SEQ ID NO: 1 and SEQ ID NO: 2 are 99.4% and 99.9%, respectively, identical to that of the hSK2 cloned from human leukemic Jurkat T cells (GenBank access No. NM_{—}021614), and 91.7% and 92.0%, respectively, identical to that of the rat rSK2 (GenBank access No. U69882). The polypeptide sequences of SEQ ID NO: 3 and SEQ ID NO: 4 are 99.4% and 99.9%, respectively, identical to the polypeptide encoded by hSK2 (GenBank protein_id: NP_{—}067627.1), and 97.4% and 97.6%, respectively, identical to that encoded by rSK2 (GenBank protein_id: AAB09563.1).

The protein products of the two clones formed functional small conductance calcium activated potassium channels in both an oocyte expression system (Example 4) or a mammalian expression system (Example 5).

### Example 4

### Functional Characterization of SK2 in an Oocyte Expression System

The clones identified in Example 3, hSK2A⁺ and hSK2A⁻, were expressed in Xenopus laevis oocytes, and function of the SK2 channels was then assessed by measuring whole cell currents as follows.

Xenopus laevis oocytes were prepared and injected using standard methods (see, Fraser et al. Electrophysiology: a practical approach. D. I. Wallis, IRL Press at Oxford University Press, Oxford: 65-86 (1993)). Briefly, ovarian lobes from adult female Xenopus laevis (Nasco, Fort Atkinson, Wis.) were teased apart, rinsed several times in nominally calcium-free saline OR-2: 82.5 mM NaCl, 2.5 mM KCl, 1 mM MgCl₂, 5 mM HEPES, adjusted to pH 7.0 with NaOH, and gently shaken in OR-2 containing 0.2% collagenase Type 1 (ICN Biomedicals, Aurora, Ohio) for 2-5 hours at 24° C. When approximately 50% of the follicular layers were removed, Stage V and VI oocytes were selected and rinsed in media consisting of 75% OR-2 and 25% ND-96. The ND-96 contained: 100 mM NaCl, 2 mM KCl, 1 mM MgCl₂, 1.8 mM CaCl₂, 5 mM HEPES, 2.5 mM Na pyruvate, gentamicin (50 μg/ml), adjusted to pH 7.0 with NaOH.

The extracellular Ca²⁺ was increased stepwise (1:4 ND96:OR-2; 1:1 and 3:1) and the cells were maintained in ND-96 for 2-24 hours before injection. For in vitro transcription, a modified PGEM HE (Liman et al., Neuron 9(5): 861-71 (1992)) containing human hSK2A⁺ or hSK2A⁻ was linearized with Nhe I restriction enzyme (New England Biolabs, Beverly, Mass.), agarose gel-purified/phenol-chloroform extracted and quantified spectrophotometrically. Linearized pGEMHE/hSK2 (300 nanograms) was used as template for T7 RNA promoter-driven in vitro transcription as per directed by the mMESSAGE mMACHINE capped mRNA transcription kit (Ambion, Austin, Tex.) protocol. Synthesized SK2 mRNA transcripts were phenol-chloroform extracted, isopropanol precipitated, washed in 80% ethanol, vacuum-dried and resuspended in nuclease-free water. SK2 mRNA transcripts were quantified spectrophotometrically and visualized by denaturing agarose gel electrophoresis (1% agarose/1M Urea) to confirm synthesis of full-length mRNA transcripts. SK2 mRNA transcripts were stored at −80° C. until further use.

Oocytes were injected with 50 nl of hSK2A⁺ orhSK2A⁻ mRNA (1-10 ng). Control oocytes were injected with 50 nl of water. Oocytes were incubated for 2 days in ND-96 before analysis for expression of human hSK2 A⁺ or hSK2A⁻. Injected oocytes were maintained in 48-well cell culture clusters (Costar; Cambridge, Mass.) at 18° C.

Whole cell currents were measured 2 days after injection with a conventional two-electrode voltage clamp (GeneClamp500, Axon Instruments, Foster City, Calif.) using standard methods previously described and known in the art (Dascal, N., CRC Crit Rev Biochem, 22(4): 317-87 (1987)). The microelectrodes were filled with 3 M KCl, which had resistances of approximately 1 MΩ. During whole cell current measurement, cells were continuously perfused with ND96 at room temperature.

Voltage protocols consisting of ramps from −130 mV to +70 mV (at a rate of 1 mV/msec) were applied to the oocyte. Bath perfusion of chlorzoxazone (1 mM), an activator for SK2 channels, for several minutes increased the outward currents measured at −25 mV similarly when applied to oocytes expressing hSK2A⁺ or hSK2A⁻.

Results indicated that hSK2A⁺ and hSK2A⁻ produced functional SK channels that can be activated by chlorzoxazone and produce current similar to known SK2 channels.

### Example 5

### Functional Characterization of SK2 in a Mammalian Expression System by Whole Cell Voltage Clamp

The clones identified in Example 3, hSK2A⁺ and hSK2A⁻, were expressed in HEK cells. Function of the SK2 channels was then assessed by measuring whole cell currents as follows.

Mammalian cell lines stably expressing hSK2 were constructed by transfecting tsA201 cells (human embryonic kidney, or HEK293, cell subclones, available commercially from Cell Genesis (Foster City, Calif.)) with a pcDNA3.1zeo expression vector (Invitrogen) containing SK2 cDNA. Transfection was performed using manufacturer's protocol (Superfect, Qiagen). Cells were maintained in Zeocin (200 μg/ml, Invitrogen) for at least a week at 37° C. to select for successfully transfected cells.

The whole cell voltage clamp technique (Hamill et al., Pflugers Arch., 391(2): 85-100 (1981)) was used to record Ca²⁺-activated K⁺ currents in cells stably expressing SK2. Cells were continuously perfused in a physiological saline (approximately 0.5 ml/min) unless otherwise indicated. The standard physiological saline (“Tyrodes”) contained: 130 mM NaCl, 4 mM KCl, 1 mM CaCl₂, 1.2 mM MgCl2, and 10 mM hemi-Na-HEPES (pH 7.3, 295-300 mOsm as measured using a Wescor 5500 vapor-pressure (Wescor, Inc., Logan, Utah)). Recording electrodes were fabricated from borosilicate capillary tubing (R6; Garner Glass, Claremont, Calif.), and had resistances of 1-2 MΩ when containing intracellular saline: 140 mM KCl, 3 mM MgCl₂, 100 ρM EGTA and 10 mM HEPES, pH 7.4. Current and voltage signals were detected and filtered at 2 kHz with an Axopatch 1D patch-clamp amplifier (Axon Instruments, Foster City, Calif.), digitally recorded with a DigiData 1200B laboratory interface (Axon Instruments) and PC compatible computer system. Data were stored on magnetic disk for off-line analysis. Data acquisition and analysis were performed with PClamp software.

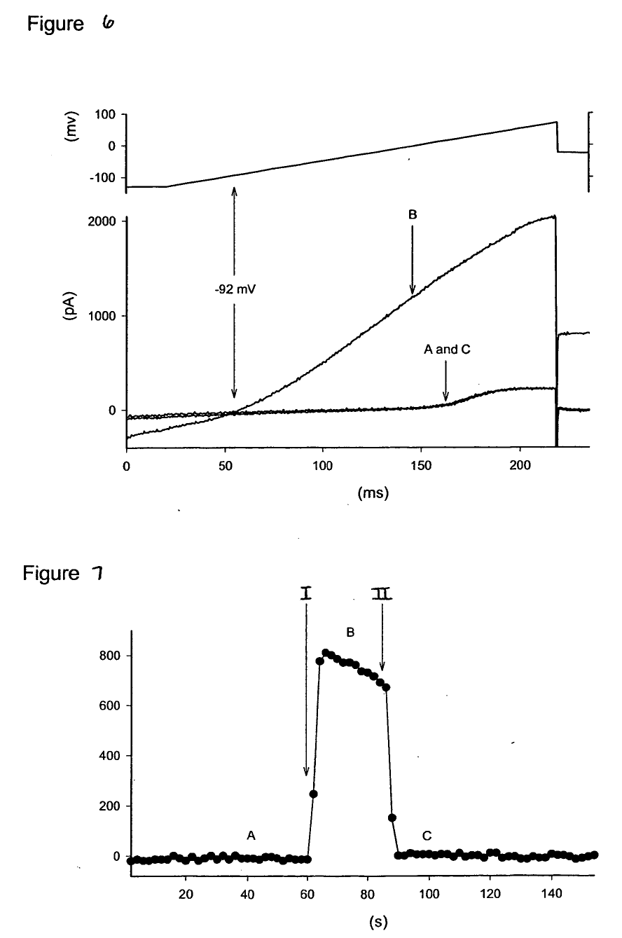

Apparent reversal potentials (Vᵣₑᵥ) of calcium-activated potassium conductances were determined using a voltage-ramp protocol [Dubin, et al., J Biol Chem 274(43): 30799-810 (1999)]. Voltage ramps were applied every 2 seconds and the resulting whole cell ramp currents were recorded. The voltage was ramped from −130 mV to 70 mV (1 mV/msec). The current required to clamp the cells at −25 mV was continuously monitored and is plotted in FIG. 6. Extracellular ATP (200 μM) and thapsigargin (1 μM) were applied to raise intracellular calcium levels and thus activate SK2 derived currents (FIG. 7).

In FIG. 6, control voltage ramp-induced currents are represented by sweep labeled “A.” Extracellular ATP and thapsigargin were added to release Ca²⁺ and activate SK2 (sweep labeled “B”). Apamin (100 nM) was subsequently applied to the cell by bath perfusion and the Ca²⁺-activated whole cell conductance was blocked (sweep labeled “C”) to control levels. For FIG. 6, time (ms) is represented on the X-axis, and current (pA) and voltage (mV) are represented on the Y-axis.

In FIG. 7, the voltage ramp-induced current traces are labeled A, B and C, where A is the whole cell current elicited prior to application of ATP/Tg, and C is the whole cell current elicited after application of 100 nM apamin to block SK2 mediated currents. In FIG. 7, ATP/Tg addition is indicated at I, and apamin addition is indicated at II.

Voltage ramp-induced currents measured in the presence of ATP and thapsigargin revealed large calcium-activated potassium currents that were subsequently blocked by apamin. The apparent reversal potential for these currents was −92 mV as predicted for a potassium current mediated by SK2 potassium channels.

The data shown in FIGS. 6 and 7 were from cells expressing hSK2 A⁺. Similar results were obtained from hSK2 A⁻expressing cells (data not shown). tsA201 cells expressing the vector pcDNA3.1 zeo without the SK2 construct did not exhibit a change in current upon ATP/Tg addition (data not shown).

Results indicate that the novel isoform, hSK2A⁺, formed functional SK2 channels in mammalian cells. Further, the SK2 channel currents increased with addition of ATP/Tg (known activator of SK2 channel activity) and were blocked by apamin (a known inhibitor of SK2 channel activity). The reversal potential for the SK2 currents was as predicted for a potassium current mediated by SK2 potassium channels.

### Example 6

### High Throughput Assay for Identifying Modulators of SK2 Channel

A tsA201 cell line stably expressing SK2 was developed as described in Example 5. These cells were plated in a black optical bottom 384 well assay plate at a density of 8×10⁶ cells/plate. A fluorescent dye sensitive to membrane potential (Molecular Devices, FLIPR Membrane Potential Kit, Cat. No. R8034) was incubated with the cells in a standard external solution (in mM: 128 NaCl, 2 CaCl₂, 2 KCl, 1 MgCl₂, 20 HEPES and dextrose added to achieve 300 mOsm, pH 7.3) according to the manufacturer's instructions. After a 30-minute incubation in the voltage sensitive dye, the cell plate was transferred to a fluorescence plate reader (FLIPR384™, Molecular Devices), and ten baseline fluorescence readings were obtained.

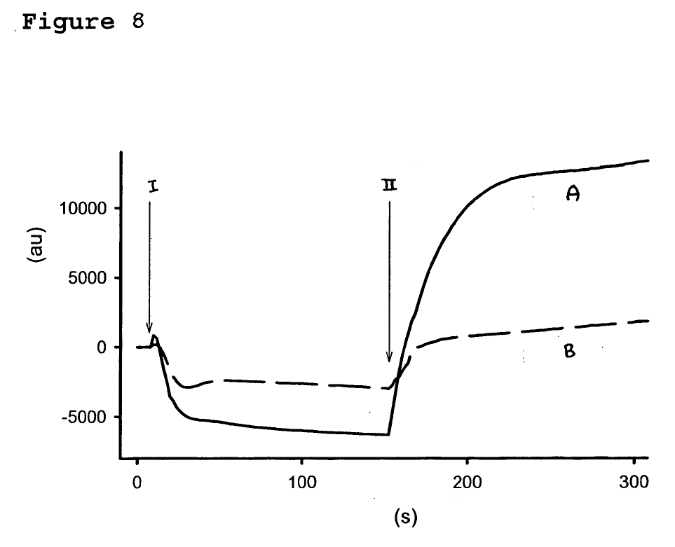

Intracellular Ca²⁺ levels of the cells were raised by addition of ATP (200 μM final concentration) and thapsigargin (1 μM final concentration). CsCl (80 mM final concentration) was subsequently added to the cells, and effect on fluorescence is illustrated in FIG. 8. In FIG. 8, time is represented in seconds on the X-axis, and fluorescence is represented in au on the Y-axis. ATP/Tg addition is indicated at time I, and CsCl addition is indicated at time II. Control is represented at curve A, and fluorescence readings upon addition of apamin is represented at curve B. As shown, the elevated intracellular Ca²⁺ evoked a large decrease in the fluorescence signal consistent with membrane potential hyperpolarization. Addition of CsCl caused a large increase in fluorescence consistent with cell depolarization, since calcium-activated K⁺ channels are permeable to Cs⁺.

Apamin was added to the cells in a final concentration of 100 nM. As shown in FIG. 8, apamin reduced both the Ca²⁺-induced decrease in fluorescence (hyperpolarization) and the Cs⁺-induced increase in fluorescence (depolarization) in cells maintained in the membrane potential sensitive dye. Data represents the average of four wells from the 384-well plate under control conditions, and the average of four wells in the presence of apamin.

Cells were then incubated for 10 minutes with compounds and subject to the ATP/thapsigargin and CsCl protocol shown in FIG. 8. The following were used in the indicated concentrations and found to have no effect on the fluorescence signals (i.e., the responses to ATP/Tg and Cs+ were similar to control signals): pentrium A (tested at 8.33 μM; inhibitor of BK channels), α-dendrotoxin (tested at 833 nM; inhibitor of some Kv channels), noxiustoxin (tested at 833 nM; inhibitor of Ca²⁺-and voltage activated channels), iberiotoxin (tested at 833 nM; inhibitor of the IK channel), and pinacidil and minoxidil (tested at 286 μM and 1.43 μM, respectively; KATP activators).

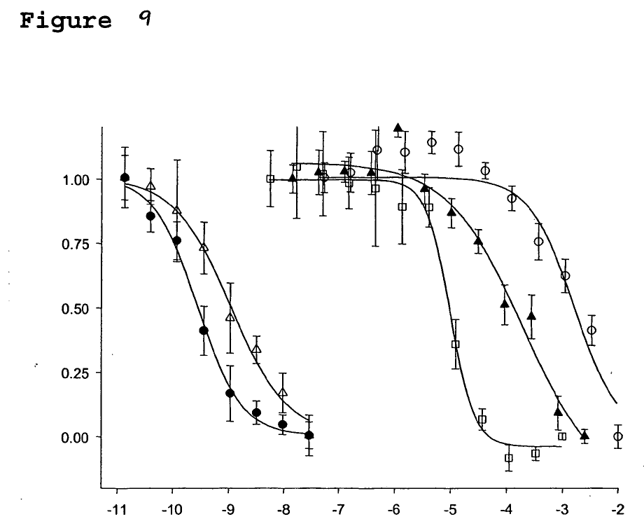

The magnitude of the ATP/Tg induced decrease in fluorescence was measured in the presence of each of a panel of known SK2 inhibitors. Each inhibitor was incubated in the reaction solution for approximately thirty (30) minutes at approximately 25° C. prior to testing on the FLIPR384™. The following inhibitors were used in the indicated concentrations (FIG. 9): apamin (tested at 0.29 nM, dark circles), scyllatoxin (tested at 1.1 nM, open triangles), NS1619 (tested at 10 μM, open squares), quinidine (tested at 200 μM, dark triangles), bicuculline methobromide (tested at 1.6 mM, open circles). Results are illustrated in FIG. 9, wherein normalized activity is represented on the Y-axis, and concentration of inhibitor (log [compound]) is represented on the X-axis. As shown, binding affinity of the inhibitors tested was as follows: apamin>scyllatoxin>NS1619>quinidine>bicuculline. The Hill coefficient (n_{H}) for each was as follows: NS16192, quinidine 0.6, bicuculline 1, apamin 1.1, scyllatoxin 0.8.

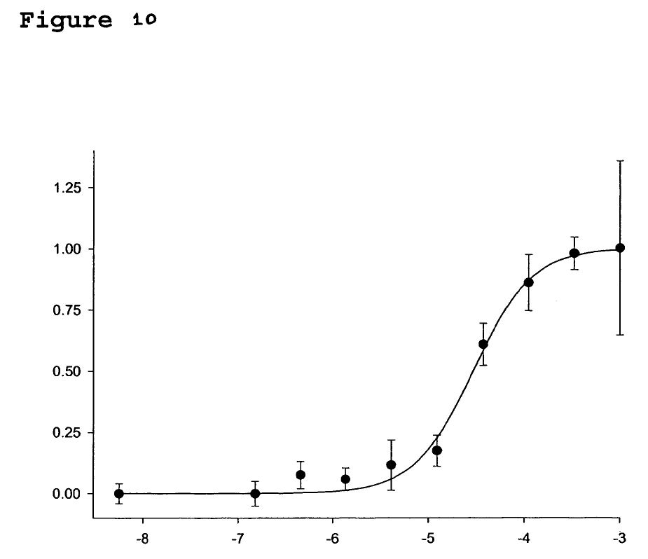

Riluzole, an opener of calcium activated potassium channels, was added online to achieve final concentrations between 100 nM and 1 mM and the resulting hyperpolarization induced by riluzole in each well was measured and plotted in FIG. 10. Results are illustrated in FIG. 10, wherein normalized activity is represented on the Y-axis, and concentration of riluzole (log [riluzole]) is represented on the X-axis. The fit of the data yielded an EC₅₀ of 29 μM with nH=1.4. Data were normalized to the maximum fluorescence achieved by riluzole.

Due to the non-linear relationship between ion flux and the resulting membrane potential change, it is desirable to screen a panel of cell lines stably expressing SK2 channels for their pharmacological profile. The most desirable cell lines are those that reveal agonist and antagonist potencies similar to the potencies observed using voltage clamp methods. For instance, cells expressing high levels of SK2 might reveal significantly left-shifted EC₅₀ values and right-shifted IC₅₀ values since only a small proportion of the channels should be activated to hyperpolarize the cell, and a large proportion of the channels should be blocked before antagonism is observed.

Methods to modify the number of functional channels can be used to improve the linearity of the assay. These methods include pharmacological (for example, essentially irreversible block by apamin during the time course of the assay), biochemical (for example, covalent modifications of amino acids), and molecular (for example, siRNA techniques).

In summary, FIGS. 9 and 10 demonstrate that this assay can generate changes in fluorescence dependent on the concentration of SK2 modulators. Inhibitors of SK2 decreased the ATP/Tg-induced decrease in fluorescence and the Cs⁺-induced increase in fluorescence (FIG. 9) and the activators (e.g., riluzole) decreased basal cell fluorescence in a dose dependent manner consistent with membrane hyperpolarization (FIG. 10). The potencies of these compounds were similar to published results [Cao et al., J Pharmacol Exp Ther., 296(3): 683-9 (2001)].

### Example 7

### Binding Assay for Identifying a Modulators of SK2 Channel

The binding of high affinity toxins, such as apamin or scyllatoxin, is useful for identifying modulators of SK2 function. These toxins are utilized in a binding assay as described in this Example.

Cells expressing SK2, such as the cell line described in Example 5 are suspended in ice-cold external solution (in mM: 130 NaCl, 2 CaCl₂, 4 MgCl₂, 10 glucose, 20 HEPES, pH 7.3) with the inclusion of 0.1% BSA at 0.5×10⁶ to 2×10⁶ cells/ml. 125I-apamin (200 μM, Dupont NEN) is then added to the cell suspension and the mixture incubated on ice for one hour with periodic gentle agitation. The mixture is centrifuged at 5,000×g for 5 minutes and the supernatant removed. Pellets are solubilized and radioactivity assessed in a gamma counter (Packard Bioscience).

Specific apamin binding is determined in the presence of 1 μM unlabeled apamin. The ability of a test molecule to compete for binding SK2 channel is studied by adding a test molecule to the binding reaction prepared above. The reaction mixtures are incubated for one hour on ice with periodic gentle agitation to achieve equilibration conditions.

Binding of [¹²⁵I] apamin is measured and compared to control (for example, [¹²⁵I] apamin binding in the absence of test molecule). Test molecules that bind SK2 channel are identified as those that enhance or inhibit ¹²⁵I-apamin binding to the SK2 expressing cells. Compounds identified in this assay can be further characterized by subjecting the compounds to assays for SK2 expression (mRNA and/or protein; see Example 1), SK2 channel activity (see Examples 2, 4, 5, and 6), and/or assays to determine binding affinity for SK2 channel or channel subunits, behavioral studies, and such other assays as are described herein.

### Example 8

### Ion Flux Assay for Identification of SK2 Channel Modulators

Ion flux are utilized in an assay to identify modulators of SK2 channels as follows.

SK2 expressing cells such as the one described in Example 5 are cultured to near confluency in a 15 cm culture plate. Culture medium is removed and replaced with fresh media containing 10 mCi/ml ⁸⁶RbCl or cold RbCl and the cells incubated at 37° C. in 5% CO₂ overnight. Culture media is aspirated and the cells are washed twice with external solution. Cells are removed from the tissue culture dish with trypsin and resuspended in external solution such as that used for electrophysiological studies at 5×10⁶ cells/ml.

To test for activators of SK2, 50 μl of the cell suspension is incubated with a small volume (5-10 μl) of test molecule in each well of a Millipore Multiscreen 96 well filter plate. Additional wells to be used for positive control contain 200 μM ATP and 1 μM thapsigargin, and the known activator riluzole at 100 μM. Negative control wells contain 100 nM apamin.

The mixture is incubated for 30 minutes at approximately 25° C. and filtered into a standard 96 well plate. In the case of the radioactive ⁸⁶Rb⁺ flux assay, the filtrate is mixed with scintillation cocktail and counted in a standard scintillation counter. Cold Rb⁺ is measured in an atomic adsorption spectrometer.

Activators of SK2 will increase Rb⁺ flux, thereby increasing presence of Rb⁺ in the reaction solution. An increase in radioactive signal, or atomic adsorption (of cold rubidium) will be measured, as compared to negative control.

To test for inhibitors of SK2, 50 μl of the cell suspension is first mixed with a small volume of test molecule. After a 10 minute incubation, a small volume of ATP and thapsigargin (200 μM and 1 μM final concentration respectively) solution is added to induce Rb⁺ efflux. In this case wells containing 20-50 nM apamin serve as a positive control.

The samples are incubated for 30 minutes, filtered and counted or tested in AAS as above (see British Journal of Pharmacology 126,1707-1716 (1999)).

Inhibitors of SK2 will decrease the amount of Rb⁺ present in the reaction solution, as compared to control containing ATP and thapsigargin. Correspondingly, a decrease in either radioactive signal or atomic adsorption will be observed.

Other embodiments of this invention will be apparent to those skilled in the art upon consideration of this specification or from practice of the invention disclosed herein. Various omissions, modifications, and changes to the principles and embodiments described herein may be made by one skilled in the art without departing from the true scope and spirit of the invention which is indicated by the following claims. All patents, patent documents, and publications cited herein are hereby incorporated by reference as if individually incorporated.

1. A method comprising steps of:
a) providing cells capable of expressing SK2;
b) contacting the cells with a test molecule;
c) obtaining information indicative of cellular SK2 expression to obtain an SK2 Expression Value;
d) comparing the SK2 Expression Value with a control SK2 Expression Value; and
e) identifying a test molecule that causes the cells to display an SK2 Expression Value that is different from the control SK2 Expression Value.

2. The method according to claim 1 wherein the identifying step comprises identifying a test molecule that causes the cells to display an SK2 Expression Value that is greater than the control SK2 Expression Value.

3. The method according to claim 2 wherein the identifying step comprises identifying a test molecule that causes the cells to display an SK2 Expression Value at least 200% greater than the control SK2 Expression Value.

4. The method according to claim 2 wherein the identifying step comprises identifying a test molecule that causes the cells to display an SK2 Expression Value at least 500% greater than the control SK2 Expression Value.

5. The method according to claim 1 wherein the step of providing cells capable of expressing SK2 comprises providing naturally-occurring SK2 expressing cells.

6. The method according to claim 1 wherein the step of providing cells capable of expressing SK2 comprises providing recombinantly modified cells.

7. The method according to claim 1 wherein the step of obtaining information indicative of cellular SK2 expression comprises analyzing SK2 mRNA expression.

8. The method according to claim 1 wherein the step of obtaining information indicative of cellular SK2 expression comprises analyzing SK2 protein expression.

9. The method according to claim 1 wherein the step of obtaining information indicative of cellular SK2 expression comprises analyzing expression of a reporter molecule.

10. A method comprising steps of:
a) providing a sample comprising a nucleic acid sequence having a gene under the control of an SK2 regulatory sequence;
b) contacting the sample with a test molecule;
c) obtaining information indicative of expression of the gene to obtain a gene Expression Value;
d) comparing the gene Expression Value with a control gene Expression Value; and
e) identifying a test molecule that causes the sample to display a gene Expression Value that is different from the control gene Expression Value.

11. The method according to claim 10 wherein the identifying step comprises identifying a test molecule that causes the sample to display a gene Expression Value that is greater than the control Expression Value.

12. The method according to claim 11 wherein the identifying step comprises identifying a test molecule that causes the sample to display a gene Expression Value at least 200% greater than the control gene Expression Value.

13. The method according to claim 11 wherein the identifying step comprises identifying a test molecule that causes the sample to display a gene Expression Value at least 500% greater than the control gene Expression Value.

14. The method according to claim 9 wherein the gene is a reporter gene.

15. The method according to claim 14 wherein the reporter gene is selected from the group consisting of genes encoding luciferase, β-galactosidase, green fluorescent protein, chloramphenicol acetyltransferase, β-glucuronidase, neomycin phosphotransferase, and guanine xanthine phosphoribosyl-transferase proteins.

16. The method according to claim 11 wherein the gene is SK2.

17. The method according to claim 14 wherein the step of obtaining information indicative of expression of the gene comprises analyzing expression of a reporter molecule.

18. The method according to claim 16 wherein the step of obtaining information indicative of expression of the gene comprises analyzing expression SK2 mRNA.

19. The method according to claim 16 wherein the step of obtaining information indicative of expression of the gene comprises analyzing expression of SK2 protein.

20. A method of identifying a molecule useful for treating neuropathic pain, the method comprising steps of:
providing cells capable of expressing SK2;
a) contacting the cells with a test molecule;
b) obtaining information indicative of SK2 cellular expression;
c) comparing the SK2 cellular expression in response to the test molecule with a control; and
d) identifying a test molecule useful for treating neuropathic pain as a molecule that causes cells to display an increase in the SK2 cellular expression relative to the control.

21. The method according to claim 20 wherein the step of providing cells capable of expressing SK2 comprises providing dorsal root ganglion cells isolated from a spinal nerve ligation animal model.

22-38. (canceled)
